(19) United States
(12) Patent Application Publication
(10) Pub. No.: US 2002/0072101 A1
(43) Pub. Date: Jun. 13, 2002

(54) Novel human nucleic acid molecules and polypeptides encoding cation channels

(76) Inventors: Glen T. Gaughan, Cheshire, CT (US); Chandra S. Ramanathan, Wallingford, CT (US)

Correspondence Address: PENNIE AND EDMONDS, 1155 AVENUE OF THE AMERICAS, NEW YORK, NY, 100362711

(21) Appl. No.: 09/764,367

(22) Filed: Jan. 18, 2001

Related U.S. Application Data
(60) Provisional application No. 60/177,554, filed on Jan. 21, 2000.

(51) Int. Cl. C12N 9/00; C07H 21/04; C12P 21/02; C12N 5/06; C07K 14/435

(52) U.S. Cl. 435/183; 435/69.1; 435/325; 435/320.1; 530/350; 536/23.2

(57) ABSTRACT

The present invention relates to novel human nucleic acid molecules encoding novel human cation channels, and proteins and polypeptides encoded by such nucleic acid molecules. More specifically, the nucleic acid molecules of the invention include novel human genes, e.g., hCCh3.1, hCCh3.2, and hCCh4, that encode proteins or polypeptides that display some sequence homology and structural homology to the vanilloid and TRP (transient receptor potential) families of cation channel proteins. The proteins and polypeptides of the invention represent novel cation channels that may be therapeutically valuable targets for drug delivery in the treatment of human diseases which involve calcium, sodium, potassium or other ionic homeostatic dysfunction, such as central nervous system (CNS) disorders, e.g., stroke or degenerative neurological disorders such as Alzheimer's disease, or other disorders such as cardiac disorders, e.g., arrhythmia, diabetes, chronic pain, hypercalcemia, hypocalcemia, hypercalciuria, hypocalciuria, or ion disorders associated with renal or liver disease.

### INTRODUCTION

The present invention relates to the isolation and identification of novel human nucleic acid molecules and proteins and polypeptides encoded by such nucleic acid molecules, or degenerate variants thereof, encoding novel human cation channels. More specifically, the nucleic acid molecules of the invention include two novel human genes encoding proteins or polypeptides that display some sequence homology and structural homology to the vanilloid and TRP (transient receptor potential) families of cation channel proteins. The proteins and polypeptides of the invention represent novel cation channels that may be therapeutically valuable targets for drug delivery in the treatment of human diseases that involve calcium, sodium, potassium or other ionic homeostatic dysfunction, such as central nervous system (CNS) disorders, e.g., stroke or degenerative neurological disorders such as Alzheimer's disease, or other disorders such as cardiac disorders, e.g., arrhythmia, diabetes, chronic pain, hypercalcemia, hypocalcemia, hypercalciuria, hypocalciuria, or ion disorders associated with renal or liver disease.

### BACKGROUND OF THE INVENTION

Control of the internal ionic environment is an extremely important function of all living cells. Ion exchange with the external medium is regulated by a variety of means, the most important of which are various transporters and ion channels. Ion channels comprise a very large and diverse family of proteins which play an important role in cell homeostasis, hormone and neurotransmitter release, motility, neuronal action potential generation and propagation and other vital intra- and inter-cellular functions. Thus, these channels are important targets for the development of therapeutic compounds in the treatment of disease.

A number of proteins have been described as forming ion channels, including the vanilloid and TRP protein families. These proteins have been shown to function as cation channels of varying degrees of selectivity and with different, and in some cases unknown, mechanisms for channel gating. For example, the TRP family of ion channels comprises a group of proteins some of which are believed to form store-operated calcium (Ca²⁺) channels, i.e., ion channels that operate to allow the influx of extracellular Ca²⁺ into cells when the intracellular stores of calcium are depleted (Zhu et al., 1996, Cell 85: 661-671). It is believed that TRP ion channels are expressed, in some form, in most, if not all, animal tissues (Zhu et al., supra at 661). Recently a human orthologue of murine Trp12 was localized on human chromosone 12 (Wisenbach et al., 2000, FEBS Letters 485:127-134). In addition, another protein, termed trp-like or trpl, has been disclosed (Phillips et al., 1992, Neuron 8: 631-642; Gillo et al., 1996, PNAS USA 93: 14146-14151) and it has been suggested that there may be a cooperative interaction between TRP and TRPL proteins, perhaps these proteins contributing channel subunits to form a multimeric Ca²⁺ channel (Gillo et al., supra).

The capsaicin receptor, also known as VR1 or vanilloid receptor subtype 1, has been isolated from rats and characterized as a Ca²⁺-permeable non-selective ion channel that is structurally related to the TRP family of ion channels (Caterina et al., 1997, Nature 389: 816-824). The rat VR1 cDNA contains an open reading frame of 2,514 nucleotides encoding a 838-amino acid protein. Hydrophilicity studies have indicated that VR1 contains six transmembrane domains with a short hydrophobic stretch between transmembrane regions 5 and 6 which may represent the ion permeation path. In addition, VR1 is disclosed as containing three ankyrin repeat domains at the N-terminal end of the protein (Caterina et al., supra at 820). It has been noted that VR1 resembles the trp and trp1 proteins in topological organization, the presence of multiple N-terminal ankyrin repeats and in amino acid sequence homology within and adjacent to the sixth transmembrane domain (Caterina et al, supra at 820-821). However, outside of these regions of homology, there is actually very little sequence homology between VR1 and the TRP-related proteins. Moreover, studies have indicated that VR1 is not a store-operated Ca²⁺ channel as are some of the TRP proteins and the expression of this protein is restricted to sensory neurons (Caterina et al., supra at 821 and FIG. 6 at 820; Mezey, E. et al., 2000, Proc. Natl. Acad. Sci. USA 97: 3655-3660).

Human VR1 (also known in the art as “hVR1” or “OTRPC1”) has been disclosed in PCT Patent Application WO 99/37675 and PCT Patent Application WO 00/29577, which disclose nucleotide and amino acid sequences for human VR1 as well as another subtype, human VR2 (also known in the art as “hVR2”, “VANILREP2”, “VRRP”, “VLR” or “OTRPC2”). In addition, PCT Patent Application WO 99/37765 discloses nucleotide and amino acid sequences for VANILREP2 and polymorphic variants thereof. The VANILREP2 protein sequence set forth in PCT application WO 99/37765 appears to be essentially the same as hVR2 disclosed in PCT application WO 99/37675. See also PCT Application WO 99/46377, which corresponds to EP 953638 A1, PCT application WO 00/22121, and GB patent application 2346882 A, which also disclose the nucleotide and amino acid sequences for hVR2.

Additional members of the vanilloid family of cation channels have also been identified. For example, a homologue of VR1, termed SIC, was cloned from the rat kidney. This protein was identified as a stretch-inactivating channel (SIC), i.e., it is inactivated by membrane stretch, and as being expressed mainly in the kidney and liver. SIC was further described as sharing the same transmembrane and pore alignments with VR1 but having different electrophysiological properties (Suzuki et al., March 1999, J. Biol. Chem. 274 (No. 10): 6330-6335). Recent reports, however, indicate that SIC may be a chimera of VR1 and a newly-identified VR subtype, OTRPC4 (see, e.g., Strotmann et al., October 2000, Nature Cell Biology 2: 695-702 and Liedtke W. et al., 2000, Cell 103: 525-535). See also Wisenbach supra, reporting that the C-terminal region of Trp12 is similar to the corresponding region of SIC. Moreover, it has been noted in the art that, despite structural homologies between members of the vanilloid family, respective proteins within the family may possess significant differences, e.g., in conductance or permeability to various ions (Suzuki et al., supra at 6335).

Another cation channel protein that has been identified as sharing a relatively low sequence homology (<30%) with the vanilloid family is ECaC (epithelial calcium channel). This protein was initially cloned from rabbit kidney cells and found to be expressed in the proximal small intestine, the kidney and the placenta of the rabbit. This protein was disclosed as resembling the VR1 and TRP family of receptors in predicted topological organization and the presence of multiple NH₂-terminal ankyrin repeats. In addition, amino acid sequence homologies between ECaC, VR1 and the TRP-related proteins were noted within and adjacent to the sixth transmembrane segment, including the predicted region for the ion permeation path (Hoenderop et al., March 1999, J. Biol. Chem. 274 (No. 13): 8375-8378). However, it was also noted that, despite these structural and sequence homologies, there is actually a low sequence homology between these proteins outside of the sixth transmembrane segment, “suggesting a distant evolutionary relationship among these channels.” (Hoenderop et al., supra at 8377).

More recently, the human homologue of ECaC, hECaC, has been identified and disclosed as having a <30% sequence homology with other Ca²⁺ channels and as being highly expressed in kidney, small intestine, and pancreas (see Muller, et al., 2000, Genomics 67: 48-53). Also, Trp12 is reported to show 32% and 46% sequence homology with the ECaC channel and the Vr1, respectively, and is highly expressed in mouse kidney (Wisenbach supra)s.

Yet another Ca²⁺ transport protein, CaT1, has been identified from rat duodenum, which protein is structurally related to the ECaC, VR1, and TRP ion channels. However, CaT1 is not stimulated by capsaicin or calcium store depletion, as would be expected with VR1 and the TRP receptors, respectively, thus suggesting that CaT1 is not a subtype of the VR1 or TRP ion channels (Peng et al., August 1999, J. Biol. Chem. 274 (No. 32): 22739-22746). More recently, a homologue of CaT1, termed CaT2, has been identified in the rat (Peng et al., September 2000, J. Biol. Chem. 275 (36): 28186-28194).

Finally, it should be noted that, while the proteins described above have clear structural and sequence homologies (compare Zhu et al., supra, FIG. 6D at 668, Caterina et al., supra, FIG. 5b at 819, and Hoenderop et al., FIG. 1B at 8376), they nevertheless display varying patterns of tissue expression, electrophysiological properties and functions (e.g., selective vs. non-selective), such that it is acknowledged in the art that these molecules, while distantly related from an evolutionary standpoint, are a diverse group of proteins with significantly different and distinct properties and functions (Suzuki et al., supra at 6335; Hoenderop et al., supra at 8377; and Caterina et al., supra at 822). For a review of the various members of this complex family of proteins, see Harteneck et al., 2000, Trends Neurosci. 23: 159-166.

### SUMMARY OF THE INVENTION

The present invention relates to the isolation and identification of novel nucleic acid molecules and proteins and polypeptides encoded by such nucleic acid molecules, or degenerate variants thereof, that participate in the formation or function of novel human ion channels. More specifically, the nucleic acid molecules of the invention include two novel human genes that encode proteins or polypeptides involved in the formation or function of novel cation channels. The novel proteins of the invention display some sequence homology and structural homology to the TRP and vanilloid family of cation channels but represent distinct human channel proteins with distinct distribution patterns, e.g., tissue expression.

According to one embodiment of the invention, a novel human cDNA, termed hCCh3, and the amino acid sequence of its derived expressed protein, is disclosed. This cDNA has been isolated in two splice forms, hCCh3.1 and hCCh3.2, which differ in the presence (hCCh3.1) or absence (hCCh3.2) of a 180 base pair segment. The encoded protein corresponding to the hCCh3.1 cDNA shows a modest level of homology to the human vanilloid receptor family of ion channels approximately 44-50%).

According to another embodiment of the invention, a novel human cDNA, termed hCCh4, and the amino acid sequence of its derived expressed protein, is disclosed. The encoded protein corresponding to the hCCh4 cDNA shows a relatively low level of homology to the human vanilloid receptor family of ion channels (approximately 30-35%). The hCCh3 and hCCh4 DNA sequences disclosed herein share an overall sequence homology of 49%.

The compositions of this invention include nucleic acid molecules, e.g., the hCCh3 and hCCh4 genes, including recombinant DNA molecules, cloned genes or degenerate variants thereof, especially naturally occurring variants, which encode novel hCCh3 and hCCh4 gene products, and antibodies directed against such gene products or conserved variants or fragments thereof.

In particular, the compositions of the present invention include nucleic acid molecules (also referred to herein as “hCCh nucleic acid molecules” or “hCCh nucleic acids”) which comprise the following sequences referred to interchangeably either “nucleotide sequences” or “nucleic acid sequences”: (a) nucleotide sequences of the human hCCh3.1, hCCh3.2 or hCCh4 genes, e.g., as depicted in FIGS. 1, 3 and 5, respectively, and as deposited with the American Type Culture Collection (ATCC) as disclosed in Section 7, infra, as well as allelic variants and homologs thereof; (b) nucleotide sequences that encode the hCCh3.1, hCCh3.2 or hCCh4 gene product amino acid sequences, as depicted in FIGS. 2, 4 and 6, respectively; (c) nucleotide sequences that encode portions of the hCCh3.1, hCCh3.2 or hCCh4 gene products corresponding to functional domains and individual exons; (d) nucleotide sequences comprising the novel hCCh3.1, hCCh3.2 or hCCh4 gene sequences disclosed herein, or portions thereof, that encode mutants of the corresponding gene product in which all or a part of one or more of the domains is deleted or altered; (e) nucleotide sequences that encode fusion proteins comprising the hCCh3.1, hCCh3.2 or hCCh4 gene product, or one or more of its domains, fused to a heterologous polypeptide; (f) nucleotide sequences within the hCCh3.1, hCCh3.2 or hCCh4 gene, as well as chromosome sequences flanking those genes, that can be utilized as part of the methods of the present invention for the diagnosis or treatment of human disease; and (g) nucleotide sequences that hybridize to the above-described sequences under stringent or moderately stringent conditions. The nucleic acid molecules of the invention include, but are not limited to, cDNA and genomic DNA sequences of the hCCh3.1, hCCh3.2 and hCCh4 genes.

The present invention also encompasses gene products of the nucleic acid molecules listed above; i.e., proteins and/or polypeptides that are encoded by the above-disclosed hCCh nucleic acid molecules, e.g., the hCCh3.1, hCCh3.2 and hCCh4 nucleic acid molecules, and are expressed in recombinant host systems.

Antagonists and agonists of the hCCh genes and/or gene products disclosed herein are also included in the present invention. Such antagonists and agonists will include, for example, small molecules, large molecules, and antibodies directed against the hCCh3.1, hCCh3.2 or hCCh4 gene product. Antagonists and agonists of the invention also include nucleotide sequences, such as antisense and ribozyme molecules, and gene or regulatory sequence replacement constructs, that can be used to inhibit or enhance expression of the disclosed hCCh nucleic acid molecules.

The present invention further encompasses cloning vectors, including expression vectors, that contain the nucleic acid molecules of the invention and can be used to express those nucleic acid molecules in host organisms. The present invention also relates to host cells engineered to contain and/or express the nucleic acid molecules of the invention. Further, host organisms that have been transformed with these nucleic acid molecules are also encompassed in the present invention, e.g., transgenic animals, particularly transgenic non-human animals, and particularly transgenic non-human mammals.

The present invention also relates to methods and compositions for the diagnosis of human disease involving cation, e.g., Ca²⁺, sodium or potassium channel, dysfunction or lack of other ionic homeostasis including but not limited to, CNS disorders such as stroke and degenerative neurological diseases, e.g., Alzheimer's disease, or disorders such as cardiac disorders, e.g., arrhythmia, diabetes, chronic pain or other disorders such as hypercalcemia, hypercalciuria, or Ca²⁺, sodium or potassium channel dysfunction that is associated with renal or liver disease. Such methods comprise, for example, measuring expression of the hCCh gene in a patient sample, or detecting a mutation in the gene in the genome of a mammal, including a human, suspected of exhibiting ion channel dysfunction. The nucleic acid molecules of the invention can also be used as diagnostic hybridization probes or as primers for diagnostic PCR analysis to identify hCCh gene mutations, allelic variations, or regulatory defects, such as defects in the expression of the gene. Such diagnostic PCR analyses can be used to diagnose individuals with disorders associated with a particular hCCh gene mutation, allelic variation, or regulatory defect. Such diagnostic PCR analyses can also be used to identify individuals susceptible to ion channel disorders.

Methods and compositions, including pharmaceutical compositions, for the treatment of ion channel disorders are also included in the invention. Such methods and compositions are capable of modulating the level of hCCh, e.g., hCCh3.1, hCCh3.2 or hCCh4, gene expression and/or the level of activity of the respective gene product. Such methods include, for example, modulating the expression of the hCCh gene and/or the activity of the hCCh gene product for the treatment of a disorder that is mediated by a defect in some other gene.

Such methods also include screening methods for the identification of compounds that modulate the expression of the nucleic acids and/or the activity of the polypeptides of the invention, e.g., assays that measure hCCh3 or hCCh4 mRNA and/or gene product levels, and assays that measure levels of hCCh3 or hCCh4 activity, such as the ability of the gene products to allow Ca²⁺ influx into cells.

For example, cellular and non-cellular assays are known that can be used to identify compounds that interact with the hCCh gene and/or gene product, e.g., modulate the activity of the gene and/or bind to the gene product. Such cell-based assays of the invention utilize cells, cell lines, or engineered cells or cell lines that express the gene product.

In one embodiment, such methods comprise contacting a compound to a cell that expresses a hCCh gene, measuring the level of gene expression, gene product expression, or gene product activity, and comparing this level to the level of the hCCh gene expression, gene product expression, or gene product activity produced by the cell in the absence of the compound, such that if the level obtained in the presence of the compound differs from that obtained in its absence, a compound that modulates the expression of the hCCh gene and/or the synthesis or activity of the gene product has been identified.

In an alternative embodiment, such methods comprise administering a compound to a host organism, e.g., a transgenic animal that expresses a hCCh transgene or a mutant hCCh transgene, and measuring the level of hCCh gene expression, gene product expression, or gene product activity. The measured level is compared to the level of hCCh gene expression, gene product expression, or gene product activity in a host that is not exposed to the compound, such that if the level obtained when the host is exposed to the compound differs from that obtained when the host is not exposed to the compound, a compound that modulates the expression of the hCCh gene and/or the synthesis or activity of hCCh gene products has been identified.

The compounds identified by these methods include therapeutic compounds that can be used as pharmaceutical compositions to reduce or eliminate the symptoms of ion channel disorders such as CNS disorders, e.g., stroke or degenerative neurological diseases, cardiac diseases or other ion-related disorders such as hypercalcemia, hypocalcemia, hypercalciuria, hypocalciuria, or ion disorders that are associated with renal or liver disease.

### DESCRIPTION OF THE FIGURES

FIG. 1. Human hCCh3.1 nucleotide sequence.

FIG. 2. Human hCCh3.1 amino acid sequence.

FIG. 3. Human hCCh3.2 nucleotide sequence.

FIG. 4. Human hCCh3.2 amino acid sequence.

FIG. 5. Human hCCh4 nucleotide sequence.

FIG. 6. Human hCCh4 amino acid sequence.

FIG. 7. Alignment of protein sequences for hCCh3 and hCCh4 with the reported vanilloid receptors VR1 and VR2. Ankyrin domains are in boldface, transmembrane domains are underlined, and the pore region is boxed.

### DETAILED DESCRIPTION OF THE INVENTION

The present invention relates to the isolation and identification of novel nucleic acid molecules and proteins and polypeptides for the formation or function of novel human ion channels. More specifically, the invention relates to novel human genes which include hCCh3.1, hCCh3.2 and hCCh4, which encode corresponding hCCh3.1, hCCh3.2 and hCCh4 proteins or biologically active derivatives or fragments thereof, involved in the formation or function of cation channels.

The “hCCh nucleic acid molecules” or “hCCh nucleic acids” of the present invention may also refer to isolated naturally-occurring or recombinantly-produced human hCCh3.1, hCCh3.2 and hCCh4 nucleic acid molecules, e.g., DNA molecules, cloned genes or degenerate variants thereof. The compositions of the invention also include isolated, naturally-occurring or recombinantly-produced human hCCh3.1, hCCh3.2 and hCCh4 proteins or polypeptides.

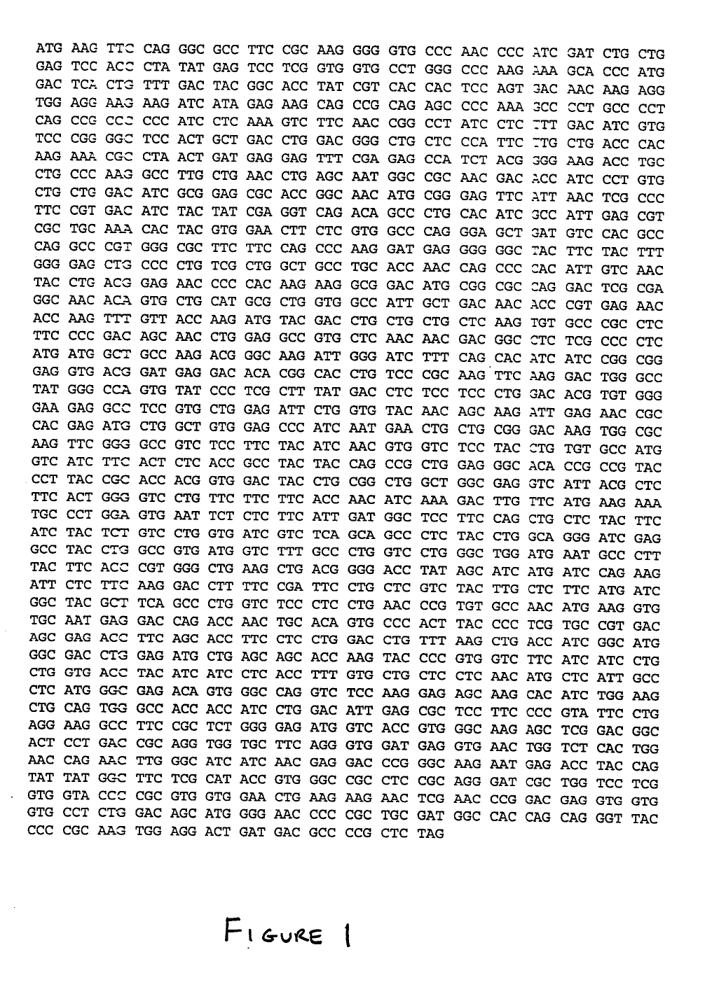

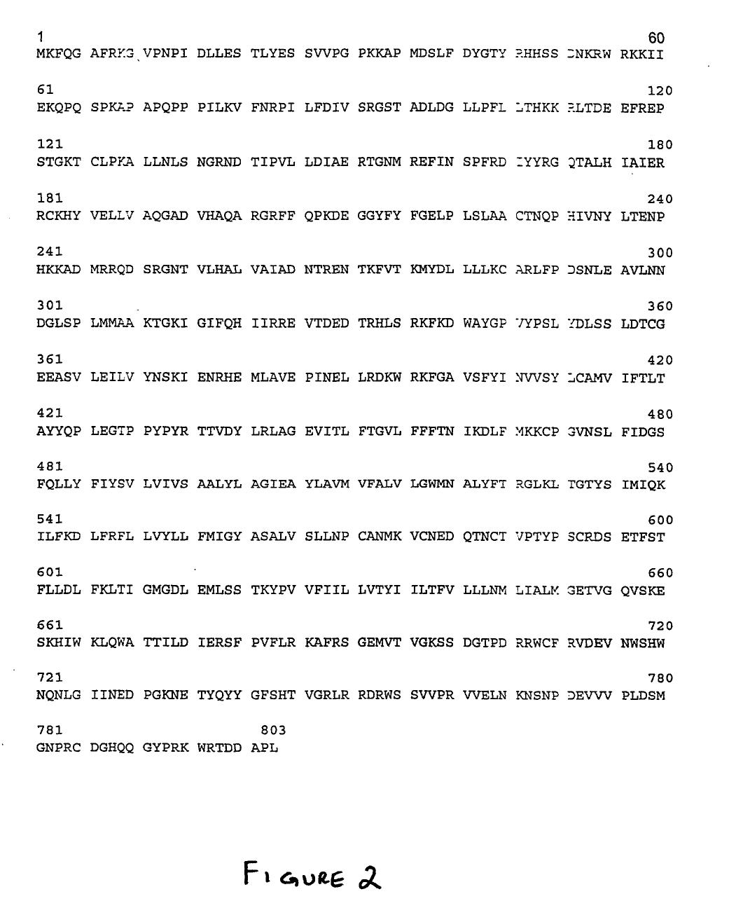

More specifically, disclosed herein is the DNA sequence of two splice variants of the hCCh3 gene of the invention. These variants are referred to herein as hCCh3.1 and hCCh3.2 (see FIGS. 1 and 3, respectively). The hCCh3.2 DNA sequence contains a deletion of 180 base pairs representing a region that includes a portion of the third ankyrin domain repeat and conserved Protein Kinase C (PKC)/calcineurin and tyrosine phosphorylation sites. The encoded protein of the hCCh3.1 gene displays a modest level of homology with the human vanilloid receptor and vanilloid-related receptor, 47% and 44% overall identity, respectively.

The DNA sequence for another hCCh gene, hCCh4, is also disclosed herein. The encoded protein of hCCh4 gene displays a relatively low level of homology with the human vanilloid receptor and vanilloid-related receptor, 33% and 31% overall identity, respectively. The hCCh3 and hCCh4 DNA sequences of this invention display an overall sequence homology of 49%. The hCCh DNA sequences and encoded proteins of this invention also differ from the vanilloid family of ion channels in their patterns of tissue expression. For example, the highest levels of hCCh3 expression occur in the trachea and salivary gland. Moderate to low levels of expression are also observed in the kidney, esophagus, mammary gland and placenta. The highest levels of hCCh4 expression occur in the placenta, prostate, salivary gland and pancreas with lower but significant levels observed in a variety of brain regions.

Moreover, neither hCCh protein channel as disclosed herein mediates the actions of capsaicin or other related vanilloids. When expressed alone, neither hCCh3 nor hCCh4 confers sensitivity to capsaicin or resiniferatoxin, ligands used in the art to define the vanilloid receptor subtypes, in a Ca²⁺-flux assay.

Other embodiments of the invention include antibodies directed to the hCCh, e.g., hCCh3.1, hCCh3.2 and hCCh4, proteins or polypeptides of the invention and methods and compositions for the diagnosis and treatment of human diseases related to ion channel dysfunction as described below.

### The hCCh Nucleic Acid Molecules of the Invention

The hCCh genes of the invention, e.g., hCCh3.1, hCCh3.2 and hCCh4, are novel human nucleic acid molecules that encode proteins or polypeptides involved in the formation or function of novel human ion channels. Although these novel genes and proteins display some sequence and structural homology to the TRP and vanilloid families of cation channel proteins as well as other cation channel proteins known in the art, it is also known in the art that proteins displaying these homologies have significant differences in function, such as conductance and permeability, as well as differences in tissue expression. As such, it is acknowledged in the art that nucleic acid molecules and the proteins encoded by those molecules sharing these homologies can still represent diverse, distinct and unique nucleic acids and proteins, respectively.

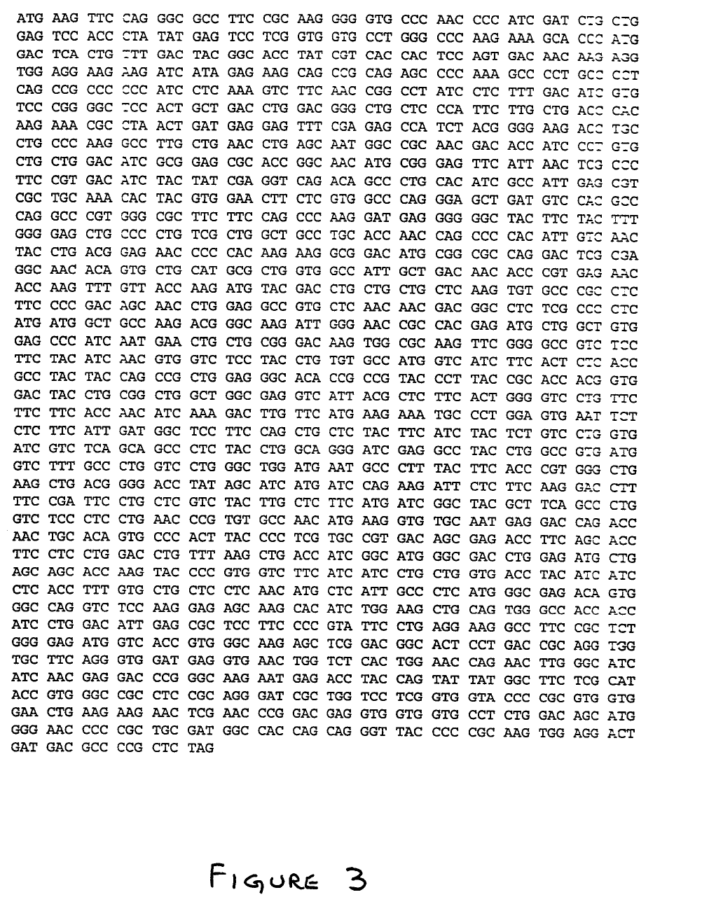

The hCCh nucleic acid molecules of the invention include the following: (a) a nucleic acid molecule containing the DNA sequence, hCCh3.1, hCCh3.2 or hCCh4, as shown in FIG. 1, 3 or 5, respectively, or as contained in the cDNA clone hCCh3.1-pcDNA3.1(+), hCCh3.2-pcDNA3.1(+), or hCCh4-pcDNA3.1(+), as deposited with the ATCC; (b) any DNA sequence that encodes the amino acid sequence, hCCh3.1, hCCh3.2 or hCCh4, as shown in FIG. 2, 4 or 6, respectively, or encoded by the cDNA clones hCCh3.1-pcDNA3.1(+), hCCh3.2-pcDNA3.1(+), or hCCh4-pcDNA3.1(+), as deposited with the ATCC; (c) any DNA sequence that hybridizes to the complement of DNA sequences that encode the amino acid sequences of FIG. 2, 4 or 6, respectively, or contained in the cDNA clones hCCh3.1-pcDNA3.1(+), hCCh3.2-pcDNA3.1(+), or hCCh4-pcDNA3.1(+), as deposited with the ATCC, under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (see, e.g., Ausubel F. M. et al., eds., 1989, Current Protocols in Molecular Biology, Vol. I, Green Publishing Associates, Inc., and John Wiley & sons, Inc., New York, at p. 2.10.3) or (d) any DNA sequence that hybridizes to the complement of DNA sequences that encode the amino acid sequences of FIG. 2, 4 or 6, respectively, or contained in the cDNA clones hCCh3.1-pcDNA3.1(+), hCCh3.2-pcDNA3.1(+), or hCCh4-pcDNA3.1(+), as deposited with the ATCC, under less stringent conditions, such as moderately stringent conditions, e.g., washing in 0.2×SSC/0.1% SDS at 42° C. (Ausubel et al., 1989, supra), and which encodes a gene product functionally equivalent to a hCCh gene product encoded by the deposited sequences or the sequences depicted in FIG. 2, 4 or 6. “Functionally equivalent” as used herein refers to any protein capable of exhibiting a substantially similar in vivo or in vitro activity as the hCCh gene products encoded by the hCCh nucleic acid molecules described herein, e.g., ion channel formation or function.

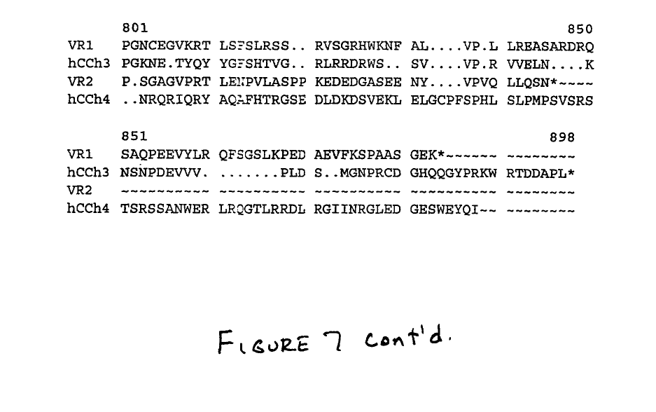

As used herein, the term “hCCh nucleic acid molecule” or “hCCh nucleic acid” may also refer to fragments and/or degenerate variants of DNA sequences (a) through (d), including naturally occurring variants or mutant alleles thereof. Such fragments include, for example, nucleic acid sequences that encode portions of the hCCh protein that correspond to functional domains of the protein. One embodiment of such a hCCh nucleic acid fragment comprises a nucleic acid that encodes the fifth and sixth transmembrane segments of the hCCh protein, including the predicted pore loop (see FIG. 7).

Additionally, the hCCh nucleic acid molecules of the invention include isolated nucleic acid molecules, preferably DNA molecules, that hybridize under highly stringent or moderately stringent hybridization conditions to at least about 6, preferably at least about 12, and more preferably at least about 18, consecutive nucleotides of the nucleic acid sequences of (a) through (d), identified supra.

The hCCh nucleic acid molecules of the invention also include nucleic acid molecules, preferably DNA molecules, that hybridize to, and are therefore complements of, the DNA sequences of (a) through (d), supra. Such hybridization conditions may be highly stringent or moderately stringent, as described above. In those instances in which the nucleic acid molecules are deoxyoligonucleotides (“oligos”), highly stringent conditions may include, e.g., washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as hCCh antisense molecules useful, for example, in hCCh gene regulation or as antisense primers in amplification reactions of hCCh nucleic acid sequences. Further, such sequences may be used as part of ribozyme and/or triple helix sequences, also useful for hCCh gene regulation. Still further, such molecules may be used as components of diagnostic methods whereby, for example, the presence of a particular hCCh allele or alternatively spliced hCCh transcript responsible for causing or predisposing one to a disorder involving ion channel dysfunction may be detected.

Typically, the hCCh nucleic acids of the invention should exhibit at least about 80% overall similarity at the nucleotide level, more preferably at least about 85-90% overall similarity and most preferably at least about 95% overall similarity to the nucleic acid sequence of FIG. 1, 3 or 5.

Also included within the hCCh nucleic acid molecules of the invention are nucleic acid molecules, preferably DNA molecules, comprising an hCCh nucleic acid, as described herein, operatively linked to a nucleotide sequence encoding a heterologous protein or peptide.

Moreover, due to the degeneracy of the genetic code, other DNA sequences that encode substantially the amino acid sequences of hCCh3.1, hCCh3.2 or hCCh4, may be used in the practice of the present invention for the cloning and expression of hCCh polypeptides. Such DNA sequences include those that are capable of hybridizing to the hCCh nucleic acids of this invention under stringent (high or moderate) conditions, or that would be capable of hybridizing under stringent conditions but for the degeneracy of the genetic code.

Altered hCCh DNA sequences that may be used in accordance with the invention include deletions, additions or substitutions of different nucleotide residues resulting in a nucleic acid molecule that encodes the same or a functionally equivalent gene product as those described supra. The gene product itself may contain deletions, additions or substitutions of amino acid residues within the hCCh protein sequence, which result in a silent change, thus producing a functionally equivalent hCCh polypeptide. Such amino acid substitutions may be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine and arginine; amino acids with uncharged polar head groups having similar hydrophilicity values include the following: leucine, isoleucine, valine; glycine, aniline; asparagine, glutamine; serine, threonine; phenylalanine, tyrosine. A functionally equivalent hCCh polypeptide can include a polypeptide which displays the same type of biological activity (e.g., cation channel) as the native hCCh protein, but not necessarily to the same extent.

The nucleic acid molecules or sequences of the invention may be engineered in order to alter the hCCh coding sequence for a variety of ends including but not limited to alterations that modify processing and expression of the gene product. For example, mutations may be introduced using techniques which are well known in the art, e.g., site-directed mutagenesis, to insert new restriction sites, to alter glycosylation patterns, phosphorylation, etc. For example, in certain expression systems such as yeast, host cells may over-glycosylate the gene product. When using such expression systems, it may be preferable to alter the hCCh coding sequence to eliminate any N-linked glycosylation sites.

In another embodiment of the invention, the hCCh nucleic acid or a modified hCCh sequence may be ligated to a heterologous sequence to encode a fusion protein. The fusion protein may be engineered to contain a cleavage site located between the hCCh sequence and the heterologous protein sequence, so that the hCCh protein can be cleaved away from the heterologous moiety.

The hCCh nucleic acid molecules of the invention can also be used as hybridization probes for obtaining hCCh cDNAs or genomic hCCh DNA. In addition, the nucleic acids of the invention can be used as primers in PCR amplification methods to isolate hCCh cDNAs and genomic DNA, e.g., from other species.

The hCCh gene sequences of the invention may also used to isolate mutant hCCh gene alleles. Such mutant alleles may be isolated from individuals either known or proposed to have a genotype related to ion channel dysfunction. Mutant alleles and mutant allele gene products may then be utilized in the screening, therapeutic and diagnostic systems described in Section 5.4., infra. Additionally, such hCCh gene sequences can be used to detect hCCh gene regulatory (e.g., promoter) defects which can affect ion channel function.

A cDNA of a mutant hCCh gene may be isolated, for example, by using PCR, a technique which is well known to those of skill in the art (see, e.g., U.S. Pat. No. 4,683,202). The first cDNA strand may be synthesized by hybridizing an oligo-dT oligonucleotide to mRNA isolated from tissue known or suspected to be expressed in an individual putatively carrying the mutant hCCh allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known in the art. By comparing the DNA sequence of the mutant hCCh allele to that of the normal hCCh allele, the mutation(s) responsible for the loss or alteration of function of the mutant hCCh gene product can be ascertained.

Alternatively, a genomic library can be constructed using DNA obtained from an individual suspected of or known to carry the mutant hCCh allele, or a cDNA library can be constructed using RNA from a tissue known, or suspected, to express the mutant hCCh allele. The normal hCCh gene or any suitable fragment thereof may then be labeled and used as a probe to identify the corresponding mutant hCCh allele in such libraries. Clones containing the mutant hCCh gene sequences may then be purified and subjected to sequence analysis according to methods well known in the art.

According to another embodiment, an expression library can be constructed utilizing cDNA synthesized from, for example, RNA isolated from a tissue known, or suspected, to express a mutant hCCh allele in an individual suspected of or known to carry such a mutant allele. Gene products made by the putatively mutant tissue may be expressed and screened using standard antibody screening techniques in conjunction with antibodies raised against the normal hCCh gene product, as described in Section 5.3, supra. For screening techniques, see, for example, Harlow, E. and Lane, eds., 1988, “Anti-bodies: A Laboratory Manual”, Cold Spring Harbor Press, Cold Spring Harbor.

In cases where a hCCh mutation results in an expressed gene product with altered function (e.g., as a result of a missense or a frameshift mutation), a polyclonal set of anti-hCCh gene product antibodies are likely to cross-react with the mutant hCCh gene product. Library clones detected via their reaction with such labeled antibodies can be purified and subjected to sequence analysis according to methods well known to those of skill in the art.

In an alternate embodiment of the invention, the coding sequence of hCCh can be synthesized in whole or in part, using chemical methods well known in the art, based on the nucleic acid and/or amino acid sequences of the hCCh genes and proteins disclosed herein. See, for example, Caruthers et al., 1980, Nuc. Acids Res. Symp. Ser. 7: 215-233; Crea and Horn, 1980, Nuc. Acids Res. 9(10): 2331; Matteucci and Caruthers, 1980, Tetrahedron Letters 21: 719; and Chow and Kempe, 1981, Nuc. Acids Res. 9(12): 2807-2817. Alternatively, the hCCh protein itself can be produced using chemical methods to synthesize the hCCh amino acid sequence in whole or in part. For example, peptides can be synthesized by solid phase techniques, cleaved from the resin, and purified by preparative high performance liquid chromatography (see, e.g., Creighton, 1983, Proteins Structures And Molecular Principles, W. H. Freeman and Co., N.Y., pp. 50-60). The composition of the synthetic peptides may be confirmed by amino acid analysis or sequencing (e.g., the Edman degradation procedure; see Creighton, 1983, Proteins, Structures and Molecular Principles, W. H. Freeman and Co., N.Y., pp. 34-49).

The invention also encompasses (a) DNA vectors that contain any of the foregoing hCCh sequences and/or their complements; (b) DNA expression vectors that contain any of the foregoing hCCh coding sequences operatively associated with a regulatory element that directs the expression of the coding sequences; and (c) genetically engineered host cells that contain any of the foregoing hCCh coding sequences operatively associated with a non-native regulatory element that directs the expression of the coding sequences in the host cell. As used herein, regulatory elements include, but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

The invention still further includes nucleic acid analogs, including but not limited to peptide nucleic acid analogues, equivalent to the nucleic acid molecules described herein. “Equivalent” as used in this context refers to nucleic acid analogs that have the same primary base sequence as the nucleic acid molecules described above. Nucleic acid analogs and methods for the synthesis of nucleic acid analogs are well known to those of skill in the art. See, e.g., Egholm, M. et al., 1993, Nature 365:566-568; and Perry-O'Keefe, H. et al., 1996, Proc. Natl. Acad. USA 93:14670-14675.

### Expression of Recombinant hCCh Polypeptides

The hCCh nucleic acid molecules of the invention may be used to generate recombinant DNA molecules that direct the expression of hCCh polypeptides, including the full-length hCCh protein, e.g., hCCh3.1, hCCh3.2, or hCCh4, functionally active or equivalent hCCh peptides thereof, or hCCh fusion proteins in appropriate host cells.

In order to express a biologically active hCCh polypeptide, a nucleic acid molecule coding for the polypeptide, or a functional equivalent thereof as described in Section 5.1, supra, is inserted into an appropriate expression vector, i.e., a vector which contains the necessary elements for the transcription and translation of the inserted coding sequence.

The hCCh gene products so produced, as well as host cells or cell lines transfected or transformed with recombinant hCCh expression vectors, can be used for a variety of purposes. These include but are not limited to generating antibodies (i.e., monoclonal or polyclonal) that bind to the hCCh protein, including those that competitively inhibit binding and thus can “neutralize” hCCh activity, and the screening and selection of hCCh analogs or ligands.

Methods which are well known to those skilled in the art are used to construct expression vectors containing the hCCh coding sequences of the invention and appropriate transcriptional and translational control signals. These methods include in vitro recombinant DNA techniques, synthetic techniques and in vivo recombination/genetic recombination. See, for example, the techniques described in Maniatis et al., 1989, Molecular Cloning, A Laboratory Manual, Cold Spring Harbor Laboratory, N.Y. and Ausubel et al., 1989, Current Protocols in Molecular Biology, Greene Publishing Associates and Wiley Interscience, N.Y. See also Sambrook et al., 1989, Molecular Cloning, A Laboratory Manual, Cold Spring Harbor Press, N.Y.

A variety of host-expression vector systems may be used to express the hCCh coding sequences of this invention. Such host-expression systems represent vehicles by which the coding sequences of interest may be produced and subsequently purified, but also represent cells which may, when transformed or transfected with the appropriate nucleotide coding sequences, exhibit the corresponding hCCh gene products in situ and/or function in vivo. These hosts include but are not limited to microorganisms such as bacteria (e.g., E. coli, B. subtilis) transformed with recombinant bacteriophage DNA, plasmid DNA or cosmid DNA expression vectors containing the hCCh coding sequences; yeast (e.g., Saccharomyces, Pichia) transformed with recombinant yeast expression vectors containing the hCCh coding sequences; insect cell systems infected with recombinant virus expression vectors (e.g., baculovirus) containing the hCCh coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the hCCh coding sequences; or mammalian cell systems (e.g., COS, CHO, BHK, 293, 3T3) harboring recombinant expression constructs containing promoters derived from the genome of mammalian cells (e.g., the metallothionein promoter) or from mammalian viruses (e.g., the adenovirus late promoter or vaccinia virus 7.5 K promoter).

The expression elements of these systems can vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of the hCCh DNA, SV40-, BPV- and EBV-based vectors may be used with an appropriate selectable marker.

In bacterial systems, a number of expression vectors may be advantageously selected depending upon the use intended for the hCCh expressed. For example, when large quantities of an hCCh polypeptide are to be produced, e.g., for the generation of antibodies or the production of the hCCh gene product, vectors which direct the expression of high 20 levels of fusion protein products that are readily purified may be desirable. Such vectors include but are not limited to the E. coli expression vector pUR278 (Ruther et al., 1983, EMBO J. 2: 1791), in which the hCCh coding sequence may be ligated into the vector in frame with the lacZ coding region so that a hybrid hCCh/lacZ protein is produced; pIN vectors (Inouye & Inouye, 1985, Nucleic Acids Res. 13: 3101-3109; Van Heeke & Schuster, 1989, J. Biol. Chem. 264: 5503-5509); and the like. pGEX vectors may also be used to express foreign polypeptides as fusion proteins with glutathione S-transferase (GST). In general, such fusion proteins are soluble and can easily be purified from lysed cells by affinity chromatography, e.g., adsorption to glutathione-agarose beads followed by elution in the presence of free glutathione. The pGEX vectors are designed to include thrombin or factor Xa protease cleavage sites so that the cloned polypeptide of interest can be released from the GST moiety. See also Booth et al., 1988, Immunol. Lett. 19: 65-70; and Gardella et al., 1990, J. Biol. Chem. 265: 15854-15859; Pritchett et al., 1989, Biotechniques 7: 580.

In yeast, a number of vectors containing constitutive or inducible promoters may be used. For a review, see Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Cold Spring Harbor Press, Vols. I and II.

In an insect system, Autocrapha californica nuclear polyhidrosis virus (AcNPV) can be used as a vector to express foreign genes. The virus grows in Spodoptera frugiperda cells. The hCCh coding sequence may be cloned into non-essential regions (for example, the polyhedrin gene) of the virus and placed under control of an AcNPV promoter (for example, the polyhedrin promoter). Successful insertion of the hCCh coding sequence will result in inactivation of the polyhedrin gene and production of non-occluded recombinant virus (i.e., virus lacking the proteinaceous coat coded for by the polyhedrin gene). These recombinant viruses can then be used to infect Spodoptera frugiperda cells in which the inserted gene is expressed (see e.g., Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the hCCh coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing hCCh in infected hosts (see, e.g., Logan & Shenk, 1984, Proc. Natl. Acad. Sci. (USA) 81: 3655-3659). Alternatively, the vaccinia 7.5 K promoter may be used (see, e.g., Mackett et al., 1982, Proc. Natl. Acad. Sci. (USA) 79: 7415-7419; Mackett et al., 1984, J. Virol. 49: 857-864; Panicali et al., 1982, Proc. Natl. Acad. Sci. 79: 4927-4931).

Specific initiation signals may also be required for efficient translation of inserted hCCh coding sequences. These signals include the ATG initiation codon and adjacent sequences. In cases where the entire hCCh gene, including its own initiation codon and adjacent sequences, is inserted into the appropriate expression vector, no additional translational control signals may be needed. However, in cases where only a portion of the hCCh coding sequence is inserted, exogenous translational control signals, including the ATG initiation codon, must be provided. Furthermore, the initiation codon must be in phase with the reading frame of the hCCh coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, WI38, etc.

For long-term, high-yield production of recombinant proteins, stable expression is preferred. For example, cell lines which stably express the hCCh polypeptides of this invention may be engineered. Thus, rather than using expression vectors which contain viral origins of replication, host cells can be transformed with hCCh nucleic acid molecules, e.g., DNA, controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express hCCh polypeptides on the cell surface. Such engineered cell lines are particularly useful in screening for hCCh analogs or ligands.

In instances where the mammalian cell is a human cell, among the expression systems by which the hCCh nucleic acid sequences of the invention can be expressed are human artificial chromosome (HAC) systems (see, e.g., Harrington et al., 1997, Nature Genetics 15: 345-355).

In another embodiment, the expression characteristics of an endogenous gene (e.g., hCCh genes) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., hCCh genes) and controls, modulates or activates. For example, endogenous hCCH genes which are normally “transcriptionally silent”, i.e., a hCCh genes which is normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous hCCh genes may be activated by insertion of a promiscuous regulatory element that works across cell types.

A heterologous regulatory element may be inserted into a stable cell line or cloned microorganism, such that it is operatively linked with and activates expression of endogenous hCCh genes, using techniques, such as targeted homologous recombination, which are well known to those of skill in the art, and described e.g., in Chappel, U.S. Pat. No. 5,272,071; PCT publication No. WO 91/06667, published May 16, 1991; Skoultchi U.S. Pat. No. 5,981,214; and Treco et al U.S. Pat. No. 5,968,502 and PCT publication No. WO 94/12650, published Jun. 9, 1994, each of which is incorporated by reference in its entirety. Alternatively, non-targeted, e.g., non-homologous recombination techniques which are well-known to those of skill in the art and described, e.g., in PCT publication No. WO 99/15650, published Apr. 1, 1999, may be used, which is incorporated by reference in its entirety.

hCCh gene products can also be expressed in transgenic animals such as mice, rats, rabbits, guinea pigs, pigs, micro-pigs, sheep, goats, and non-human primates, e.g., baboons, monkeys, and chimpanzees. The term “transgenic” as used herein refers to animals expressing hCCh nucleic acid sequences from a different species (e.g., mice expressing human hCCh nucleic acid sequences), as well as animals that have been genetically engineered to overexpress endogenous (i.e., same species) hCCh nucleic acid sequences or animals that have been genetically engineered to no longer express endogenous hCCh nucleic acid sequences (i.e., “knock-out” animals), and their progeny.

Transgenic animals according to this invention may be produced using techniques well known in the art, including but not limited to pronuclear microinjection (Hoppe, P.C. and Wagner, T. E., 1989, U.S. Pat. No.4,873,191); retrovirus mediated gene transfer into germ lines (Van der Putten et al., 1985, Proc. Natl. Acad. Sci., USA 82: 6148-6152); gene targeting in embryonic stem cells (Thompson et al., 1989, Cell 56: 313-321); electroporation of embryos (Lo, 1983, Mol Cell. Biol. 3: 1803-1814); and sperm-mediated gene transfer (Lavitrano et al., 1989, Cell 57: 717-723); etc. For a review of such techniques, see Gordon, 1989, Transgenic Animals, Intl. Rev. Cytol. 115: 171-229.

In addition, any technique known in the art may be used to produce transgenic animal clones containing a hCCh transgene, for example, nuclear transfer into enucleated oocytes of nuclei from cultured embryonic, fetal or adult cells induced to quiescence (Campbell et al., 1996, Nature 380: 64-66; Wilmut et al., 1997, Nature 385: 810-813).

Host cells which contain the hCCh coding sequence and which express a biologically active gene product may be identified by at least four general approaches; (a) DNA-DNA or DNA-RNA hybridization; (b) the presence or absence of “marker” gene functions; (c) assessing the level of transcription as measured by the expression of hCCh mRNA transcripts in the host cell; and (d) detection of the gene product as measured by immunoassay or by its biological activity.

In the first approach, the presence of the hCCh coding sequence inserted in the host cell can be detected by DNA-DNA or DNA-RNA hybridization using probes comprising nucleotide sequences that are homologous to the hCCh coding sequence, respectively, or portions or derivatives thereof.

In the second approach, the selection of host cells that have been engineered to overexpress the endogenous gene (e.g., by targeted or non-targeted insertion of expression control elements) may be accomplished by DNA-RNA hybridization, e.g., Northern analysis. Expression host systems can be identified and selected based upon the presence or absence of certain “marker” gene functions. For example, if the hCCh coding sequence is inserted within a marker gene sequence of the vector, recombinants containing the hCCh coding sequence can be identified by the absence of the marker gene function. Alternatively, a marker gene can be placed in tandem with the hCCh sequence under the control of the same or different promoter used to control the expression of the hCCh coding sequence. Expression of the marker in response to induction or selection indicates expression of the hCCh coding sequence.

Selectable markers include resistance to antibiotics, resistance to methotrexate, transformation phenotype, and occlusion body formation in baculovirus. In addition, thymidine kinase activity (Wigler et al., 1977, Cell 11: 223) hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) genes can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77: 3567; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); neo, which confers resistance to the aminoglycoside G-418 (Colberre-Garapin, et al., 1981, J. Mol. Biol. 150: 1); and hygro, which confers resistance to hygromycin (Santerre et al., 1984, Gene 30: 147). Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85: 8047); and ODC (ornithine decarboxylase) which confers resistance to the ornithine decarboxylase inhibitor, 2-(difluoromethyl)-DL-ornithine, DFMO (McConlogue, 1987, in Current Communications in Molecular Biology, Cold Spring Harbor Laboratory ed.).

In the third approach, transcriptional activity for the hCCh coding region can be assessed by hybridization assays. For example, RNA can be isolated and analyzed by Northern blot using a probe homologous to the hCCh coding sequence or particular portions thereof. Alternatively, total nucleic acids of the host cell may be extracted and assayed for hybridization to such probes.

In the fourth approach, the expression of the hCCh protein product can be assessed immunologically, for example by Western blots, immunoassays such as radioimmuno-precipitation, enzyme-linked immunoassays and the like. The ultimate test of the success of the expression system, however, involves the detection of biologically active hCCh gene product. A number of assays can be used to detect hCCh activity including but not limited to binding assays and biological assays for hCCh activity.

Once a clone that produces high levels of a biologically active hCCh polypeptide is identified, the clone may be expanded and used to produce large amounts of the polypeptide which may be purified using techniques well known in the art, including but not limited to, immunoaffinity purification using antibodies, immunoprecipitation or chromatographic methods including high performance liquid chromatography (HPLC).

Where the hCCh coding sequence is engineered to encode a cleavable fusion protein, purification may be readily accomplished using affinity purification techniques. For example, a collagenase cleavage recognition consensus sequence may be engineered between the carboxy terminus of hCCh and protein A. The resulting fusion protein may be readily purified using an IgG column that binds the protein A moiety. Unfused hCCh may be readily released from the column by treatment with collagenase. Another example would be the use of pGEX vectors that express foreign polypeptides as fusion proteins with glutathionine S-transferase (GST). The fusion protein may be engineered with either thrombin or factor Xa cleavage sites between the cloned gene and the GST moiety. The fusion protein may be easily purified from cell extracts by adsorption to glutathione agarose beads followed by elution in the presence of glutathione. In fact, any cleavage site or enzyme cleavage substrate may be engineered between the hCCh gene product sequence and a second peptide or protein that has a binding partner which could be used for purification, e.g., any antigen for which an immunoaffinity column can be prepared.

In addition, hCCh fusion proteins may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

### Antibodies to hCCh Polypeptides

The present invention also includes antibodies directed to the hCCh polypeptides of this invention and methods for the production of those antibodies, including antibodies that specifically recognize one or more hCCh epitopes or epitopes of conserved variants or peptide fragments of hCCh.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a hCCh protein or polypeptide in an biological sample and may, therefore, be utilized as part of a diagnostic or prognostic technique whereby patients may be tested for abnormal levels of hCCh and/or for the presence of abnormal forms of the protein. Such antibodies may also be utilized in conjunction with, for example, compound screening protocols for the evaluation of the effect of test compounds on hCCh levels and/or activity. Additionally, such antibodies can be used in conjunction with the gene therapy techniques described in Section 5.4, infra, to, for example, evaluate normal and/or genetically-engineered hCCh-expressing cells prior to their introduction into the patient.

For the production of antibodies against hCCh, various host animals may be immunized by injection with the protein or a portion thereof. Such host animals include rabbits, mice, rats, and baboons. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a hCCh polypeptide, or an antigenic functional derivative thereof. For the production of polyclonal antibodies, host animals such as those described above, may be immunized by injection with the hCCh polypeptide supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein (1975, Nature 256: 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridomas producing the monoclonal antibodies of this invention may be cultivated in vitro or in vivo.

In addition, techniques developed for the production of chimeric antibodies (Morrison et al., 1984, Proc. Natl. Acad. Sci., 81: 6851-6855; Neuberger et al., 1984, Nature 312: 604-608; Takeda et al., 1985, Nature 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397.)

In addition, techniques have been developed for the production of humanized antibodies (see, e.g., Queen, U.S. Pat. No. 5,585,089). Humanized antibodies are antibody molecules from non-human species having one or more CDRs from the non-human species and a framework region from a human immunoglobulin molecule.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be used in the production of single chain antibodies against hCCh. Single chain antibodies are formed by linking the heavy and light chain fragments of the Fv region via an amino acid bridge, resulting in a single chain polypeptide.

Furthermore, antibody fragments which recognize specific epitopes of hCCh may be produced by techniques well known in the art. For example, such fragments include but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed (Huse et al., 1989, Science 246: 1275-1281) to allow rapid and easy identification of monoclonal Fab fragments with the desired specificity.

### Uses of the hCCh Nucleic Acid Molecules, Gene Products and Antibodies

As discussed supra, the hCCh genes of this invention encode proteins that are involved in the formation or function of ion channels, more particularly, cation channels. Given the importance of cations such as calcium, sodium or potassium in many cellular processes, the hCCh nucleic acid molecules and polypeptides of this invention are useful for the diagnosis and treatment of a variety of human disease conditions which involve ion, more particularly, cation, channel dysfunction.

For example, calcium plays a role in the release of neurotransmitters, hormones and other circulating factors, the expression of numerous regulatory genes as well as the cellular process of apoptosis or cell death. Potassium provides for neuroprotection and also affects insulin secretion. Sodium is involved in the regulation of normal neuronal action potential generation and propagation. Sodium channel blockers such as lidocaine are important analgesics. Therefore, cation channel dysfunction may play a role in many human diseases and disorders such as CNS disorders, e.g., stroke or Alzheimer's disease, and other diseases such as cardiac disorders, e.g., arrhythmia, diabetes, chronic pain, hypercalcemia, hypercalciuria, or ion channel dysfunction that is associated with renal or liver disease. As such, proteins that are involved in either the formation or function of these ion channels and the nucleic acids that encode those proteins) are useful for the diagnosis and treatment of many human diseases.

Among the uses for the nucleic acid molecules and polypeptides of the invention are the prognostic and diagnostic evaluation of human disorders involving ion/cation channel dysfunction, and the identification of subjects with a predisposition to such disorders, as described below. Other uses include methods for the treatment of such ion/cation channel dysfunction disorders, for the modulation of hCCh gene-mediated activity, and for the modulation of hCCh-mediated effector functions.

In addition, the nucleic acid molecules and polypeptides of the invention can be used in assays for the identification of compounds which modulate the expression of the hCCh genes of the invention and/or the activity of the hCCh gene products. Such compounds can include, for example, other cellular products or small molecule compounds that are involved in cation homeostasis or activity.

Diagnosis and Prognosis of Ion-Related Disorders

Methods of the invention for the diagnosis and prognosis of human diseases involving ion, e.g., cation, dysfunction may utilize reagents such as the hCCh nucleic acid molecules and sequences described in Sections 5.1, supra, or antibodies directed against hCCh polypeptides, including peptide fragments thereof, as described in Section 5.3., supra. Specifically, such reagents may be used, for example, for: (1) the detection of the presence of hCCh gene mutations, or the detection of either over- or under-expression of hCCh gene mRNA relative to the non-cation dysfunctional state or the qualitative or quantitative detection of alternatively spliced forms of hCCh transcripts which may correlate with certain ion homeostasis disorders or susceptibility toward such disorders; and (2) the detection of either an over- or an under-abundance of hCCh gene product relative to the non-cation dysfunctional state or the presence of a modified (e.g., less than full length) hCCh gene product which correlates with a cation dysfunctional state or a progression toward such a state.

The methods described herein may be performed, for example, by utilizing pre-packaged diagnostic test kits comprising at least one specific hCCh gene nucleic acid or anti-hCCh gene antibody reagent described herein, which may be conveniently used, e.g., in clinical settings, to screen and diagnose patients exhibiting ion/cation channel/homeostasis abnormalities and to screen and identify those individuals exhibiting a predisposition to such abnormalities.

For the detection of hCCh mutations, any nucleated cell can be used as a starting source for genomic nucleic acid. For the detection of hCCh transcripts or hCCh gene products, any cell type or tissue in which the hCCh gene is expressed may be utilized.

Nucleic acid-based detection techniques are described in Section 5.4.1.1., infra, whereas peptide-based detection techniques are described in Section 5.4.1.2., infra.

Detection of hCCh Gene Nucleic Acid Molecules

Mutations or polymorphisms within the hCCh gene can be detected by utilizing a number of techniques. Nucleic acid from any nucleated cell can be used as the starting point for such assay techniques, and may be isolated according to standard nucleic acid preparation procedures which are well known to those of skill in the art.

Genomic DNA may be used in hybridization or amplification assays of biological samples to detect abnormalities involving hCCh gene structure, including point mutations, insertions, deletions and chromosomal rearrangements. Such assays may include, but are not limited to, direct sequencing (Wong, C. et al., 1987, Nature 330:384-386), single stranded conformational polymorphism analyses (SSCP; Orita, M. et al., 1989, Proc. Natl. Acad. Sci. USA 86:2766-2770), heteroduplex analysis (Keen, T. J. et al., 1991, Genomics 11:199-205; Perry, D. J. & Carrell, R. W., 1992), denaturing gradient gel electrophoresis (DGGE; Myers, R. M. et al., 1985, Nucl. Acids Res. 13:3131-3145), chemical mismatch cleavage (Cotton, R. G. et al., 1988, Proc. Natl. Acad. Sci. USA 85:4397-4401) and oligonucleotide hybridization (Wallace, R. B. et al., 1981, Nucl. Acids Res. 9:879-894; Lipshutz, R. J. et al., 1995, Biotechniques 19:442-447).

Diagnostic methods for the detection of hCCh gene specific nucleic acid molecules, in patient samples or other appropriate cell sources, may involve the amplification of specific gene sequences, e.g., by PCR, followed by the analysis of the amplified molecules using techniques well known to those of skill in the art, such as, for example, those listed above. Utilizing analysis techniques such as these, the amplified sequences can be compared to those which would be expected if the nucleic acid being amplified contained only normal copies of the hCCh gene in order to determine whether a hCCh gene mutation exists.

Further, well-known genotyping techniques can be performed to type polymorphisms that are in close proximity to mutations in the hCCh gene itself. These polymorphisms can be used to identify individuals in families likely to carry mutations. If a polymorphism exhibits linkage disequilibrium with mutations in the hCCh gene, it can also be used to identify individuals in the general population likely to carry mutations. Polymorphisms that can be used in this way include restriction fragment length polymorphisms (RFLPs), which involve sequence variations in restriction enzyme target sequences, single-base polymorphisms and simple sequence repeat polymorphisms (SSLPs).

For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within the hCCh gene, and the diagnosis of diseases and disorders related to hCCh mutations.

Also, Caskey et al. (U.S. Pat. No. 5,364,759) describe a DNA profiling assay for detecting short tri- and tetra-nucleotide repeat sequences. The process includes extracting the DNA of interest, such as the hCCh gene, amplifying the extracted DNA, and labelling the repeat sequences to form a genotypic map of the individual's DNA.

A hCCh probe could additionally be used to directly identify RFLPs. Additionally, a hCCh probe or primers derived from the hCCh sequences of the invention could be used to isolate genomic clones such as YACs, BACs, PACs, cosmids, phage or plasmids. The DNA contained in these clones can be screened for single-base polymorphisms or simple sequence length polymorphisms (SSLPs) using standard hybridization or sequencing procedures.

Alternative diagnostic methods for the detection of hCCh gene-specific mutations or polymorphisms can include hybridization techniques which involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including the hCCh nucleic acid molecules of the invention including recombinant DNA molecules, cloned genes or degenerate variants thereof, as described in Section 5.1 supra, under conditions favorable for the specific annealing of these reagents to their complementary sequences within the hCCh gene. Preferably, the lengths of these nucleic acid reagents are at least 15 to 30 nucleotides. After incubation, all non-annealed nucleic acids are removed from the nucleic acid:hCCh molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid molecules of the invention as described in Section 5.1 are easily removed. Detection of the remaining, annealed, labeled hCCh nucleic acid reagents is accomplished using standard techniques well-known to those in the art. The hCCh gene sequences to which the nucleic acid molecules of the invention have annealed can be compared to the annealing pattern expected from a normal hCCh gene sequence in order to determine whether a hCCh gene mutation is present.

Quantitative and qualitative aspects of hCCh gene expression can also be assayed. For example, RNA from a cell type or tissue known, or suspected, to express the hCCh gene may be isolated and tested utilizing hybridization or PCR techniques as described supra. The isolated cells can be derived from cell culture or from a patient. The analysis of cells taken from culture may be a necessary step in the assessment of cells to be used as part of a cell-based gene therapy technique or, alternatively, to test the effect of compounds on the expression of the hCCh gene. Such analyses may reveal both quantitative and qualitative aspects of the expression pattern of the hCCh gene, including activation or inactivation of hCCh gene expression and presence of alternatively spliced hCCh transcripts.

In one embodiment of such a detection scheme, a cDNA molecule is synthesized from an RNA molecule of interest (e.g., by reverse transcription of the RNA molecule into cDNA). All or part of the resulting cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the hCCh nucleic acid molecules of the invention as described in Section 5.1, supra. The preferred lengths of such nucleic acid reagents are at least 9-30 nucleotides.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Such RT-PCR techniques can be utilized to detect differences in hCCh transcript size which may be due to normal or abnormal alternative splicing. Additionally, such techniques can be utilized to detect quantitative differences between levels of full length and/or alternatively spliced hCCh transcripts detected in normal individuals relative to those individuals exhibiting ion dysfunction disorders or exhibiting a predisposition to toward such disorders.

In the case where detection of specific alternatively spliced species is desired, appropriate primers and/or hybridization probes can be used, such that, in the absence of such sequence, no amplification would occur. Alternatively, primer pairs may be chosen utilizing the sequences depicted in FIG. 1, 3 or 5 to choose primers which will yield fragments of differing size depending on whether a particular exon is present or absent from the hCCh transcript being utilized.

As an alternative to amplification techniques, standard Northern analyses can be performed if a sufficient quantity of the appropriate cells can be obtained. Utilizing such techniques, quantitative as well as size-related differences between hCCh transcripts can also be detected.

Additionally, it is possible to perform hCCh gene expression assays in situ, i.e., directly upon tissue sections (fixed and/or frozen) of patient tissue obtained from biopsies or resections, such that no nucleic acid purification is necessary. The nucleic acid molecules of the invention as described in Section 5.1 may be used as probes and/or primers for such in situ procedures (see, for example, Nuovo, G. J., 1992, “PCR In Situ Hybridization: Protocols And Applications”, Raven Press, NY).

### Detection of hCCh Gene Products

Antibodies directed against wild type or mutant hCCh gene products or conserved variants or peptide fragments thereof as described supra may also be used for the diagnosis and prognosis of ion or cation-related disorders. Such diagnostic methods may be used to detect abnormalities in the level of hCCh gene expression or abnormalities in the structure and/or temporal, tissue, cellular, or subcellular location of hCCh gene products. Antibodies, or fragments of antibodies, may be used to screen potentially therapeutic compounds in vitro to determine their effects on hCCh gene expression and hCCh peptide production. The compounds which have beneficial effects on ion and cation-related disorders can be identified and a therapeutically effective dose determined.

In vitro immunoassays may be used, for example, to assess the efficacy of cell-based gene therapy for ion or cation-related disorders. For example, antibodies directed against hCCh peptides may be used in vitro to determine the level of hCCh gene expression achieved in cells genetically engineered to produce hCCh peptides. Such analysis will allow for a determination of the number of transformed cells necessary to achieve therapeutic efficacy in vivo, as well as optimization of the gene replacement protocol.

The tissue or cell type to be analyzed will generally include those which are known, or suspected, to express the hCCh gene. The protein isolation methods employed may, for example, be such as those described in Harlow, E. and Lane, D., 1988, “Antibodies: A Laboratory Manual”, Cold Spring Harbor Laboratory Press, Cold Spring Harbor, N.Y. The isolated cells can be derived from cell culture or from a patient. The analysis of cells taken from culture may be a necessary step in the assessment of cells to be used as part of a cell-based gene therapy technique or, alternatively, to test the effect of compounds on the expression of the hCCh gene.

Preferred diagnostic methods for the detection of hCCh gene products or conserved variants or peptide fragments thereof, may involve, for example, immunoassays wherein the hCCh gene products or conserved variants, including gene products which are the result of alternatively spliced transcripts, or peptide fragments are detected by their interaction with an anti-hCCh gene product-specific antibody.

For example, antibodies, or fragments of antibodies, such as those described in Section 5.3 supra, may be used to quantitatively or qualitatively detect the presence of hCCh gene products or conserved variants or peptide fragments thereof. The antibodies (or fragments thereof) may, additionally, be employed histologically, as in immunofluorescence or immunoelectron microscopy, for in situ detection of hCCh gene products or conserved variants or peptide fragments thereof. In situ detection may be accomplished by removing a histological specimen from a patient, and applying thereto a labeled hCCh antibody of the present invention. The antibody (or fragment) is preferably applied by overlaying the labeled antibody (or fragment) onto a biological sample. Through the use of such a procedure, it is possible to determine not only the presence of the hCCh gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for hCCh gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying hCCh gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins, The support may then be washed with suitable buffers followed by treatment with the detectably labeled hCCh gene specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-hCCh gene product antibody may be determined according to well known methods. Those skilled in the art will be able to determine operative and optimal assay conditions for each determination by employing routine experimentation.

One of the ways in which the hCCh gene peptide-specific antibody can be detectably labeled is by linking the antibody to an enzyme in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect hCCh gene peptides through the use of a radioimmunoassay (RIA) (see, for example, Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### Screening Assays for Compounds That Modulate hCCh Activity

Screening assays can be used to identify compounds that modulate hCCh activity. These compounds can include, but are not limited to, peptides, small organic or inorganic molecules or macromolecules such as nucleic acid molecules or proteins, and may be utilized, e.g., in the control of ion and cation-related disorders, in the modulation of cellular processes such as the release of neurotransmitters or other cellular regulatory factors, cell activation or regulation, cell death and changes in cell membrane properties. These compounds may also be useful, e.g., in elaborating the biological functions of hCCh gene products, modulating those biological functions and for ameliorating symptoms of ion or cation-related disorders.

The compositions of the invention include pharmaceutical compositions comprising one or more of these compounds. Such pharmaceutical compositions can be formulated as discussed in Section 5.5, infra.

More specifically, these compounds can include compounds that bind to hCCh gene products, compounds that bind to other proteins that interact with a hCCh gene product and/or interfere with the interaction of the hCCh gene product with other proteins, and compounds that modulate the activity of the hCCh gene, i.e., modulate the level of hCCh gene expression and/or modulate the level of hCCh gene product activity.

For example, assays may be utilized that identify compounds that bind to hCCh gene regulatory sequences, e.g., promoter sequences (see e.g., Platt, K. A., 1994, J. Biol. Chem. 269:28558-28562), which compounds may modulate the level of hCCh gene expression. In addition, functional assays can be used to screen for compounds that modulate hCCh gene product activity. In such assays, compounds are screened for agonistic or antagonistic activity with respect to a biological activity or function of the hCCh gene product, such as changes in the intracellular levels of an ion or cation, changes in regulatory factor release, or other activities or functions of the hCCh polypeptides of the invention.

According to a preferred embodiment, a Ca²⁺ flux assay can be utilized to monitor calcium uptake in hCCh-expressing host cells. The host cells are pre-loaded with a Ca²⁺-sensitive fluorescently-labeled dye (e.g., Fluo-4, Fluo-3, Indo-1 or Fura-2), i.e., the intracellular calcium is fluorescently labelled with the dye, and the effect of the compound, e.g., on the intracellular levels of the labeled-calcium determined and compared to the intracellular levels of control cells, e.g., lacking exposure to the compound of interest. Compounds that have an agonistic, i.e., stimulatory, modulatory effect on hCCh activity are those that, when contacted with the hCCh-expressing cells, produce an increase in intracellular calcium relative to the control cells, whereas those compounds having an antagonistic modulatory effect on hCCh activity will be those that produce a decrease in intracellular calcium. A Ca²⁺ flux assay is exemplified in Example Section 6.1, infra.

Functional assays for monitoring the effects of compounds on the levels or flux of other ions can be similarly performed; for example, the levels of potassium can be monitored using rubidium influx.

Screening assays may also be designed to identify compounds capable of binding to the hCCh gene products of the invention. Such compounds may be useful, e.g., in modulating the activity of wild type and/or mutant hCCh gene products, in elaborating the biological function of the hCCh gene product, and in screens for identifying compounds that disrupt normal hCCh gene product interactions, or may in themselves disrupt such interactions.

The principle of such screening assays to identify compounds that bind to the hCCh gene product involves preparing a reaction mixture of the hCCh gene product and the test compound under conditions and for a time sufficient to allow the two components to interact with, i.e., bind to, and thus form a complex, which can represent a transient complex, which can be removed and/or detected in the reaction mixture. For example, one assay involves anchoring a hCCh gene product or the test substance onto a solid phase and detecting hCCh gene product/test compound complexes anchored on the solid phase at the end of the reaction. In one embodiment of such a method, the hCCh gene product may be anchored onto a solid surface, and the test compound, which is not anchored, may be labeled, either directly or indirectly.

The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for hCCh gene product or the test compound to anchor any complexes formed in solution, and a labeled antibody specific for the other component of the possible complex to detect anchored complexes.

Compounds that modulate hCCh gene product activity can also include compounds that bind to proteins that interact with the hCCh gene product. These modulatory compounds can be identified by first identifying those proteins that interact with the hCCh gene product, e.g., by standard techniques known in the art for detecting protein-protein interactions, such as co-immunoprecipitation, crosslinking and co-purification through gradients or chromatographic columns. Utilizing procedures such as these allows for the isolation of proteins that interact with hCCh gene products or polypeptides of the invention as described supra.

Once isolated, such a protein can be identified and can, in turn, be used, in conjunction with standard techniques, to identify additional proteins with which it interacts. For example, at least a portion of the amino acid sequence of the protein that interacts with the hCCh gene product can be ascertained using techniques well known to those of skill in the art, such as via the Edman degradation technique (see, e.g., Creighton, 1983, “Proteins: Structures and Molecular Principles”, W. H. Freeman & Co., N.Y., pp.34-49). The amino acid sequence thus obtained may be used as a guide for the generation of oligonucleotide mixtures that can be used to screen for gene sequences encoding such proteins. Screening may be accomplished, for example, by standard hybridization or PCR techniques. Techniques for the generation of oligonucleotide mixtures and screening are well-known (see, e.g., Ausubel, supra., and PCR Protocols: A Guide to Methods and Applications, 1990, Innis, M. et al., eds. Academic Press, Inc., New York).

Additionally, methods may be employed that result in the simultaneous identification of genes which encode proteins interacting with hCCh gene products or polypeptides. These methods include, for example, probing expression libraries with labeled hCCh protein, using hCCh protein in a manner similar to the well known technique of antibody probing of λgt11 libraries. One method that detects protein interactions in vivo is the two-hybrid system. A version of this system in described by Chien et al., 1991, Proc. Natl. Acad. Sci. USA, 88:9578-9582 and is commercially available from Clontech (Palo Alto, Calif.).

In addition, compounds that disrupt hCCh interactions with its interacting or binding partners, as determined immediately above, may be useful in regulating the activity of the hCCh gene product, including mutant hCCh gene products. Such compounds may include, but are not limited to molecules such as peptides, and the like, which may bind to the hCCh gene product as described above.

The basic principle of the assay systems used to identify compounds that interfere with the interaction between the hCCh gene product and its interacting partner or partners involves preparing a reaction mixture containing the hCCh gene product, and the interacting partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of hCCh gene product and its interacting partner. Control reaction mixtures are incubated without the test compound or with a placebo. The formation of any complexes between the hCCh gene product and the interacting partner is then detected. The formation of a complex in the control reaction, but not in the reaction mixture containing the test compound, indicates that the compound interferes with the interaction of the hCCh gene product and the interacting partner. Additionally, complex formation within reaction mixtures containing the test compound and a normal hCCh gene product may also be compared to complex formation within reaction mixtures containing the test compound and a mutant hCCh gene product. This comparison may be important in those cases wherein it is desirable to identify compounds that disrupt interactions of mutant but not normal hCCh proteins.

The assay for compounds that interfere with the interaction of hCCh gene products and interacting partners can be conducted in a heterogeneous or homogeneous format. Heterogeneous assays involve anchoring either the hCCh gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the hCCh gene products and the interacting partners, e.g., by competition, can be identified by conducting the reaction in the presence of the test substance; i.e., by adding the test substance to the reaction mixture prior to or simultaneously with the hCCh gene product and interacting partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the hCCh gene product or the interacting partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the hCCh gene product or interacting partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the interacting components to anchor any complexes formed in solution, and a labeled antibody specific for the other partner to detect anchored complexes. Again, depending upon the order of addition of reactants to the liquid phase, test compounds that inhibit complex formation or that disrupt preformed complexes can be identified.

In an alternate embodiment, a preformed complex of the hCCh gene protein and the interacting partner is prepared in which either the hCCh gene product or its interacting partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt hCCh gene protein/interacting partner interaction can be identified.

In another embodiment of the invention, these same techniques can be employed using peptide fragments that correspond to the binding domains of the hCCh protein and/or the interacting partner, in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interacting, e.g., binding. Alternatively, one protein can be anchored to a solid surface using methods described in this Section above, and allowed to interact with, e.g., bind, to its labeled interacting partner, which has been treated with a proteolytic enzyme, such as trypsin. After washing, a short, labeled peptide comprising the interacting, e.g.,binding, domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

### Methods and Compositions for the Treatment of Ion Channel-Related Disorders

The present invention also relates to methods and compositions for the treatment or modulation of any disorder or cellular process that is mediated or regulated by hCCh gene product expression or function, e.g., hCCh-mediated cell activation, signal transduction, cellular regulatory factor release, etc. Further, hCCh effector functions can be modulated via such methods and compositions.

The methods of the invention include methods that modulate hCCh gene and gene product activity. In certain instances, the treatment will require an increase, upregulation or activation of hCCh activity, while in other instances, the treatment will require a decrease, downregulation or suppression of hCCh activity. “Increase” and “decrease” refer to the differential level of hCCh activity relative to hCCh activity in the cell type of interest in the absence of modulatory treatment. Methods for the decrease of hCCh activity are discussed in Section 5.4.3.1, infra. Methods for the increase of hCCh activity are discussed in Section 5.4.3.2, infra. Methods which can either increase or decrease hCCh activity depending on the particular manner in which the method is practiced are discussed in Section 5.4.3.3, infra.

### Methods for Decreasing hCCh Activity

Successful treatment of ion channel/ionic homeostasis disorders, e.g., CNS disorders, cardiac disorders or hypercalcemia, can be brought about by methods which serve to decrease hCCh activity. Activity can be decreased by, e.g., directly decreasing hCCh gene product activity and/or by decreasing the level of hCCh gene expression.

For example, compounds such as those identified through assays described in Section 5.4.2., supra, that decrease hCCh gene product activity can be used in accordance with the invention to ameliorate symptoms associated with ion channel/ionic homeostasis disorders. As discussed supra, such molecules can include, but are not limited to peptides, including soluble peptides, and small organic or inorganic molecules, and can be referred to as hCCh antagonists. Techniques for the determination of effective doses and administration of such compounds are described in Section 5.5., infra.

In addition, antisense and ribozyme molecules that inhibit hCCh gene expression can also be used to reduce the level of hCCh gene expression, thus effectively reducing the level of hCCh gene product present, thereby decreasing the level of hCCh activity. Still further, triple helix molecules can be utilized in reducing the level of hCCh gene expression. Such molecules can be designed to reduce or inhibit either wild type, or if appropriate, mutant target gene activity. Techniques for the production and use of such molecules are well known to those of skill in the art.

Antisense approaches involve the design of oligonucleotides (either DNA or RNA) that are complementary to hCCh gene mRNA. The antisense oligonucleotides will bind to the complementary hCCh gene mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See generally, Wagner, R., 1994, Nature 372:333-335. Thus, oligonucleotides complementary to either the 5′- or 3′-non-translated, non-coding regions of, e.g., the hCCh3.1, hCCh3.2 and hCCh4 genes, as depicted in FIG. 1, 3 and 5, respectively, could be used in an antisense approach to inhibit translation of endogenous hCCh gene mRNA.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of target or pathway gene mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and non-specific biological effects of oligonucleotides. It is also preferred that these studies compare levels of the target RNA or protein with that of an internal control RNA or protein. Additionally, results obtained using the antisense oligonucleotide are preferably compared with those obtained using a control oligonucleotide. It is preferred that the control oligonucleotide is of approximately the same length as the antisense oligonucleotide and that the nucleotide sequence of the control oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc.

The oligonucleotide may also include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Application No. WO 88/09810) or the blood-brain barrier (see, e.g., PCT Application No. WO 89/10134), or hybridization-triggered cleavage agents (see, e.g., Krol et al., 1988, BioTechniques 6:958-976) or intercalating agents (see, e.g., Krol, 1988, Pharm. Res. 5:539-549). For example, the oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209) and methylphosphonate oligonucleotides can be prepared by use of controlled pore glass polymer supports (Sarin et al., 1988, Proc. Natl. Acad. Sci. U.S.A. 85:7448-7451), etc.

The antisense molecules should be delivered to cells which express the hCCh gene in vivo. A number of methods have been developed for delivering antisense DNA or RNA to cells; e.g., antisense molecules can be injected directly into the tissue site or modified antisense molecules designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Thus, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous hCCh gene transcripts and thereby prevent translation of the hCCh gene mRNA. For example, a vector can be introduced in vivo such that it is taken up by a cell and directs the transcription of an antisense RNA.

Ribozymes are enzymatic RNA molecules capable of catalyzing the specific cleavage of RNA (For a review, see, e.g., Rossi, J., 1994, Current Biology 4:469-471). The mechanism of ribozyme action involves sequence-specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins.

Ribozyme molecules designed to catalytically cleave hCCh gene mRNA transcripts can also be used to prevent translation of hCCh gene mRNA and expression of target or pathway genes. (See, e.g., PCT Application No. WO 90/11364; Sarver et al., 1990, Science 247:1222-1225).

The ribozymes of the present invention also include RNA endoribonucleases (hereinafter referred to as “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; PCT Patent Application No. WO 88/04300; Been and Cech, 1986, Cell, 47:207-216). The Cech-type ribozymes have an eight base pair active site which hybridizes to a target RNA sequence, after which cleavage of the target RNA takes place. The invention encompasses those Cech-type ribozymes which target eight base-pair active site sequences that are present in an hCCh gene.

As in the antisense approach, the ribozymes can be composed of modified oligonucleotides (e.g. for improved stability, targeting, etc.) and should be delivered to cells which express the hCCh gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous hCCh gene messages and inhibit translation. Because ribozymes, unlike antisense molecules, are catalytic, a lower intracellular concentration is required for efficiency.

Endogenous hCCh gene expression can also be reduced by inactivating or “knocking out” the target and/or pathway gene or its promoter using targeted homologous recombination (see, e.g., Smithies et al., 1985, Nature 317:230-234; Thomas & Capecchi, 1987, Cell 51:503-512; Thompson et al., 1989 Cell 5:313-321). For example, a mutant, non-functional hCCh gene (or a completely unrelated DNA sequence) flanked by DNA homologous to the endogenous hCCh gene (either the coding regions or regulatory regions of the hCCh gene) can be used, with or without a selectable marker and/or a negative selectable marker, to transfect cells that express the hCCh gene in vivo. Insertion of the DNA construct, via targeted homologous recombination, results in inactivation of the hCCh gene. Such techniques can also be utilized to generate ion/cation disorder animal models. It should be noted that this approach can be adapted for use in humans provided the recombinant DNA constructs are directly administered or targeted to the required site in vivo using appropriate viral vectors, e.g., herpes virus vectors.

Alternatively, endogenous hCCh gene expression can be reduced by targeting deoxyribonucleotide sequences complementary to the regulatory region of the hCCh gene (i.e., the hCCh gene promoter and/or enhancers) to form triple helical structures that prevent transcription of the hCCh gene in target cells in the body (see generally, Helene, C., 1991, Anticancer Drug Des. 6(6):569-84; Helene, C., et al., 1992, Ann. N.Y. Acad. Sci. 660:27-36; and Maher, L. J., 1992, Bioassays 14(12):807-15).

Nucleic acid molecules to be used in triple helix formation for the inhibition of transcription should be single stranded and composed of deoxynucleotides. The base composition of these oligonucleotides should be designed to promote triple helix formation via Hoogsteen base pairing rules, which generally require sizeable stretches of either purines or pyrimidines to be present on one strand of the duplex. Nucleotide sequences may be pyrimidine-based, which will result in TAT and CGC+triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands of the triplex.

Alternatively, the potential sequences that can be targeted for triple helix formation may be increased by creating a “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of the duplex.

In instances wherein the antisense, ribozyme, and/or triple helix molecules described herein are utilized to inhibit mutant hCCh gene expression, it is possible that the technique may so efficiently reduce or inhibit the transcription (triple helix) and/or translation (antisense, ribozyme) of mRNA produced by normal target gene alleles that the concentration of normal target gene product present may be lower than is necessary for a normal phenotype. In such cases, to ensure that substantially normal levels of hCCh gene activity are maintained, nucleic acid molecules that encode and express hCCh gene polypeptides exhibiting normal target gene activity can be introduced into cells via gene therapy methods that do not contain sequences susceptible to whatever antisense, ribozyme, or triple helix treatments are being utilized. In instances where the target gene encodes an extracellular protein, it can be preferable to coadminister normal target gene protein in order to maintain the requisite level of target gene activity.

Antisense RNA and DNA, ribozyme, and triple helix molecules of the invention can be prepared by any method known in the art, e.g., methods for chemically synthesizing oligodeoxyribonucleotides and oligoribonucleotides well known in the art such as solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

In addition, well-known modifications to DNA molecules can be introduced into the hCCh nucleic acid molecules of the invention as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

### Methods for Increasing hCCh Activity

Successful treatment of ion/cation disorders can also be brought about by techniques which serve to increase the level of hCCh activity. Activity can be increased by, for example, directly increasing hCCh gene product activity and/or by increasing the level of hCCh gene expression.

For example, compounds such as those identified through the assays described in Section 5.4.2., supra, that increase hCCh activity can be used to treat ion/cation-related disorders. Such molecules can include, but are not limited to peptides, including soluble peptides, and small organic or inorganic molecules, and can be referred to as hCCh agonists.

For example, a compound can, at a level sufficient to treat ion/cation-related disorders and symptoms, be administered to a patient exhibiting such symptoms. One of skill in the art will readily know how to determine the concentration of effective, non-toxic doses of the compound, utilizing techniques such as those described infra.

Alternatively, in instances wherein the compound to be administered is a peptide compound, DNA sequences encoding the peptide compound can be directly administered to a patient exhibiting an ion/cation-related disorder or symptoms, at a concentration sufficient to produce a level of peptide compound sufficient to ameliorate the symptoms of the disorder. Any of the techniques discussed infra, which achieve intracellular administration of compounds, such as, for example, liposome administration, can be utilized for the administration of such DNA molecules. In the case of peptide compounds which act extracellularly, the DNA molecules encoding such peptides can be taken up and expressed by any cell type, so long as a sufficient circulating concentration of peptide results for the elicitation of a reduction in the ion/cation disorder symptoms.

In cases where the ion/cation disorder can be localized to a particular portion or region of the body, the DNA molecules encoding such modulatory peptides may be administered as part of a delivery complex. Such a delivery complex can comprise an appropriate nucleic acid molecule and a targeting means. Such targeting means can comprise, for example, sterols lipids, viruses or target cell specific binding agents. Viral vectors can include, but are not limited to adenovirus, adeno-associated virus, and retrovirus vectors, in addition to other particles that introduce DNA into cells, such as liposomes.

Further, in instances wherein the ion/cation-related disorder involves an aberrant hCCh gene, patients can be treated by gene replacement therapy. One or more copies of a normal hCCh gene or a portion of the gene that directs the production of a normal hCCh gene protein with hCCh gene function, can be inserted into cells, via, for example a delivery complex as described supra.

Such gene replacement techniques can be accomplished either in vivo or in vitro. Techniques which select for expression within the cell type of interest are preferred. For in vivo applications, such techniques can, for example, include appropriate local administration of hCCh gene sequences.

Additional methods which may be utilized to increase the overall level of hCCh activity include the introduction of appropriate hCCh gene-expressing cells, preferably autologous cells, into a patient at positions and in numbers which are sufficient to ameliorate the symptoms of the ion/cation-related disorder. Such cells may be either recombinant or non-recombinant. Among the cells which can be administered to increase the overall level of hCCh gene expression in a patient are normal cells, which express the hCCh gene. The cells can be administered at the anatomical site of expression, or as part of a tissue graft located at a different site in the body. Such cell-based gene therapy techniques are well known to those skilled in the art (see, e.g., Anderson, et al., U.S. Pat. No. 5,399,349; Mulligan and Wilson, U.S. Pat. No. 5,460,959).

hCCh gene sequences can also be introduced into autologous cells in vitro. These cells expressing the hCCh gene sequence can then be reintroduced, preferably by intravenous administration, into the patient until the disorder is treated and symptoms of the disorder are ameliorated.

### Additional Modulatory Techniques

The present invention also includes modulatory techniques which, depending on the specific application for which they are utilized, can yield either an increase or a decrease in hCCh activity levels leading to the amelioration of ion/cation-related disorders such as those described above.

Antibodies exhibiting modulatory capability can be utilized according to the methods of this invention to treat the ion/cation-related disorders. Depending on the specific antibody, the modulatory effect can be an increase or decrease in hCCh activity. Such antibodies can be generated using standard techniques described in Section 5.3, supra, against full length wild type or mutant hCCh proteins, or against peptides corresponding to portions of the proteins. The antibodies include but are not limited to polyclonal, monoclonal, Fab fragments, single chain antibodies, chimeric antibodies, etc.

Lipofectin or liposomes can be used to deliver the antibody or a fragment of the Fab region which binds to the hCCh gene product epitope to cells expressing the gene product. Where fragments of the antibody are used, the smallest inhibitory fragment which binds to the hCCh protein's binding domain is preferred. For example, peptides having an amino acid sequence corresponding to the domain of the variable region of the antibody that binds to the hCCh protein can be used. Such peptides can be synthesized chemically or produced via recombinant DNA technology using methods well known in the art (e.g., see Creighton, 1983, supra and Sambrook et al., 1989, supra). Alternatively, single chain antibodies, such as neutralizing antibodies, which bind to intracellular epitopes can also be administered. Such single chain antibodies can be administered, for example, by expressing nucleotide sequences encoding single-chain antibodies within the target cell population by utilizing, for example, techniques such as those described in Marasco et al., 1993, Proc. Natl. Acad. Sci. USA 90:7889-7893.

### Pharmaceutical Preparations and Methods of Administration

The compounds, e.g., nucleic acid sequences, polypeptides, peptides, and recombinant cells, described supra can be administered to a patient at therapeutically effective doses to treat or ameliorate ion/cation-related disorders. A therapeutically effective dose refers to that amount of a compound or cell population sufficient to result in amelioration of the disorder symptoms, or alternatively, to that amount of a nucleic acid sequence sufficient to express a concentration of hCCh gene product which results in the amelioration of the disorder symptoms.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the methods of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. It is preferred that hCCh-expressing cells be introduced into patients via intravenous administration.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

### EXAMPLE

### Identification of two Novel hCCH Genes and Their Encoded Proteins

The section below describes the identification of novel human gene sequences encoding novel human ion channels.

### Cloning of Novel hCCh3 DNA Sequences

In general all routine molecular biology procedures followed standard protocols or relied on widely available commercial kits and reagents. All sequencing was done with an ABI 373 automated sequencer using commercial dye-terminator chemistry.

The published rat vanilloid receptor (VR1) sequence was used in a homology search (BLAST) of expressed sequence tag EST databases, both public (e.g. NCBI) and private (Incyte Pharmaceuticals, Inc. LifeSeq Database). The selected EST set was further analyzed and overlapping sequences identified and assembled to provide a limited number of longer contiguous sequences (“contigs”). These were sorted by their degree of homology to the reported rat channel. None of the contigs included an entire coding sequence. Some of the contigs were overlapping but not identical and thus represented segments of independent genes; others were non-overlapping and thus could have represented fragments of the same or different genes.

Complete gene sequence data was subsequently obtained as follows:

hCCh3.1. Two EST clones were selected which appeared to contain inserts spanning the largest portion of the candidate gene from a database set comprising a contig not included in previously reported genes. The physical clones were obtained from Incyte. Complete sequencing of these clones provided part but not all of the coding sequence. One of the EST clones included a portion of an alternatively-spliced form, designated hCCh3.2, which variant is defined by a deletion of 180 base pairs (60 amino acids). The region deleted includes a portion of a third ankyrin domain repeat and conserved PKC/calcineurin and tyrosine phosphorylation sites which are likely to be important regulatory sites.

The remaining upstream sequence was obtained from kidney cDNA (Clontech Marathon-Ready cDNA) using the 5′ RACE method, following the manufacturer's recommended protocol using a gene-specific primer (see, e.g., Bertling, W. M. et al., 1993, PCR Methods Appl. 3: 95-99 and Frohman, M. A., 1991, Methods in Enzymology 218: 340-362).

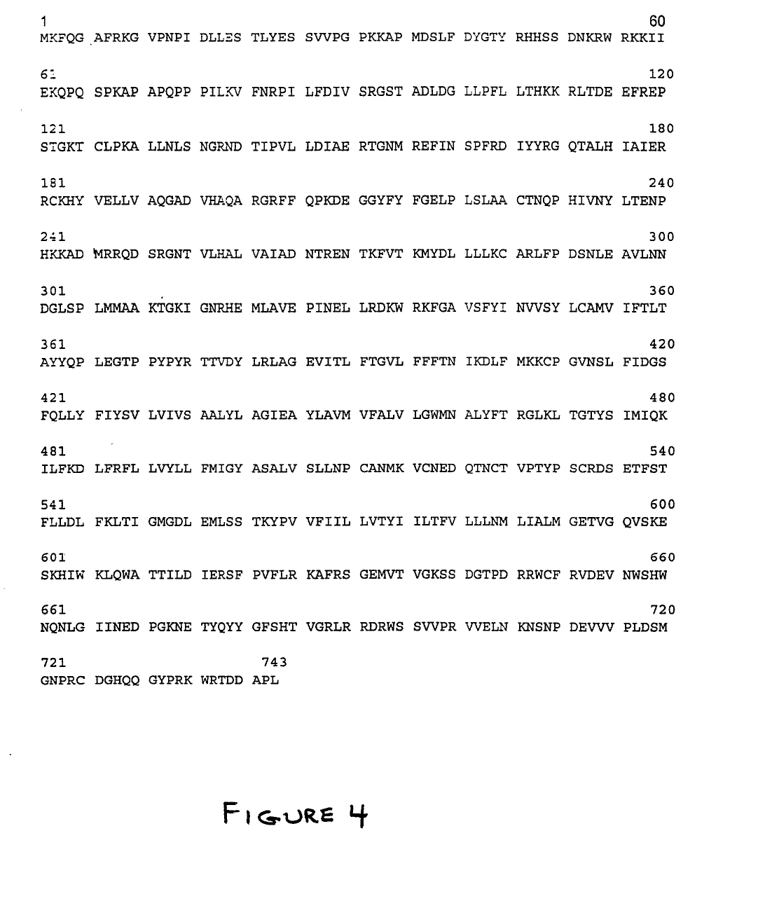

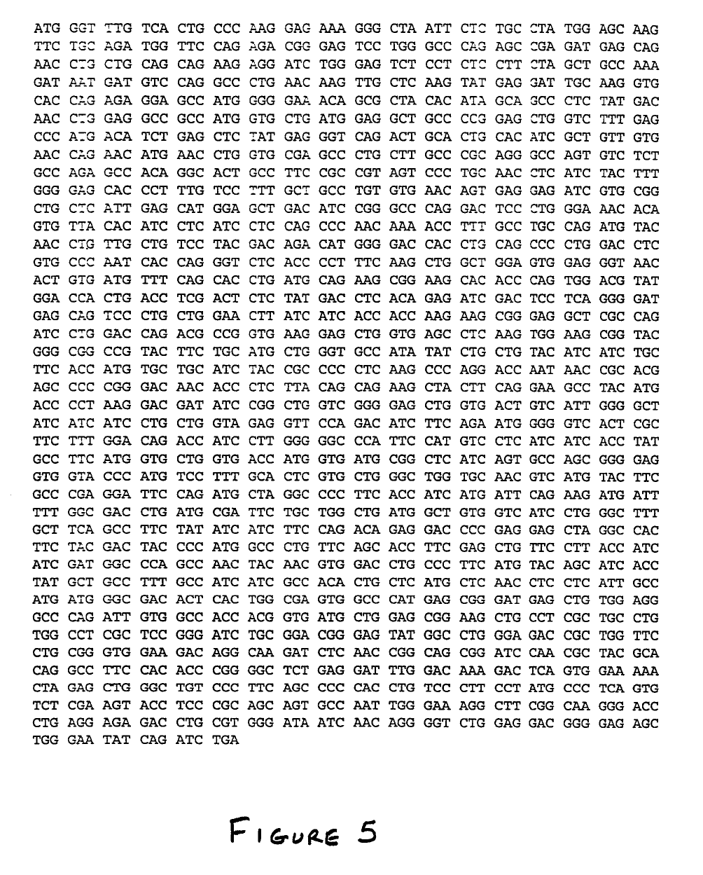

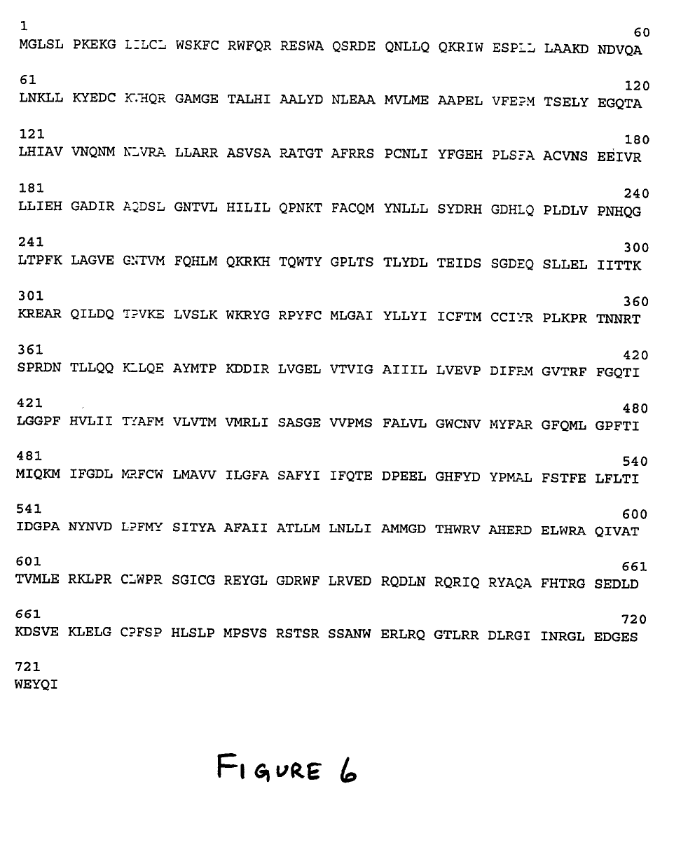

The DNA sequences for hCCh3.1 and hCCh3.2 are depicted in FIGS. 1 and 3, respectively. The derived protein, i.e., amino acid, sequences encoded by the hCCh3.1 and hCCh3.2 genes are depicted in FIGS. 2 and 4, respectively.

For expression, the PCR-derived hCCh3.1 splice form was selected and the gene was amplified from the Clontech cDNA using a forward primer which included a Bam HI restriction site and Kozak consensus sequence (TTG GAT CCA CCA TGA AGT TCC AGG GCG CCT TCC GCA) and a reverse primer which included an Xba I restriction site (TTT CTA GAC TAG AGC GGG GCG TCA TCA GTC). The gene was then subcloned into the commercially-available expression plasmid, pcDNA3.1(+) (Invitrogen).

In addition, for ease of detection of hCCh gene expression, a construct coding for hCCh3.1 fused at the N-terminus to a fluorescent protein variant (“enhanced yellow fluorescent protein”) was further prepared by subcloning hCCh3.1 directly into the commercially-available expression vector, pEYFP-CI (Clontech). This construct was termed hCCh3.1-pEYFP-C1.

Furthermore, a part of the EST clone including the alternatively-spliced segment was subcloned into the hCCh3.1-pcDNA3.1(+) and hCCh3.1-pEYFP-C1 constructs using available restriction sites (Sph I and EcoR I) to produce the hChh3.2-pcDNA3.1(+) and hCCh3.2-pEYFP-C1 constructs of the invention.

The expression patterns of the hCCh3 genes were determined using Human Multiple Tissue Northern Blots and Multiple Tissue Expression Assays obtained from Clontech. A cDNA probe, derived from a ca. 600 bp Apa I-Kpn I fragment from the 3′ UTR of hCCh3 which is common to both splice forms, was radiolabeled with [α-³²P]dCTP (3000 Ci/mmol) using the DecaPrime II DNA labeling kit (Ambion). This segment represented a region of low homology between hCCh3 and the VR and trp channel proteins, providing a probe of the highest specificity. Control blots confirmed the lack of cross hybridization. Prehybridization and hybridization (16 h; 65° C.; ˜10⁶ CPM probe per mL) in ExpressHybe buffer (Clontech) generally followed protocols recommended by the manufacturer. Blots were washed twice at high stringency (0.1×SSC/0.1% SDS; 65°)and film autoradiograms were exposed for 18-24 h and ca. 4d.

These experiments indicated that, with the exception of the trachea and salivary gland, expression of the hCCh3 gene is low to weak in organ tissues. These results do not, however, rule out high levels of expression within defined cell types that may comprise a small portion of any of the examined tissues.

In addition, a Ca²⁺-flux assay was performed to determine the effect on hCCh3 of various ligands known to affect the vanilloid proteins. More specifically, Ca²⁺ uptake was measured in transiently transfected CHO cells, i.e., transfected with the hCCh nucleic acid molecules of the invention, using the Ca²⁺-sensitive dye Fluo-4 (Molecular Probes) in a Molecular Devices Fluorometric Imaging Plate Reader (FLIPR). Cells were loaded with the dye for 30-90 minutes prior to the experiment in the presence of sulfinpyrazone. Test reagents were added, and Ca²⁺ uptake measured over a three minute period.

In these experiments, hCCh3 did not confer sensitivity to capsaicin or resiniferatoxin, two ligands used to define the vanilloid receptor subtypes. Thus, the novel human channels encoded by hCCh3 do not appear to mediate their actions via capsaicin or other related vanilloids.

The complete sequence for hCCh3 can be identified in a set of sequences from a large genomic fragment (AC007834) reported as part of the human genome sequencing project. Sequencing of the latter is as yet incomplete and the reported sequences not yet assembled into a verified contig or the sequence described herein reported as a distinct gene.

### Cloning of a Novel hCCh4 DNA Sequence

The hCCh4 DNA sequence of the invention was obtained essentially as described above, i.e., the rat VR1 sequence was used to search for homologous sequences in EST databases. PCR primers were prepared using data from a contig not included in previously reported genes. These were used to screen human prostate cDNA (Invitrogen); a positive result indicated that hCCh4 is expressed therein. The database consensus sequence was extended in the 5′ direction by the RACE procedure using prostate cDNA as template (Clontech Marathon-Ready cDNA). For expression, the entire gene was amplified from the Invitrogen cDNA using a forward primer which included a EcoR 1 restriction site and Kozak consensus sequence (CGA AU CTA CCA TGG GTT TGT CAC TGC CCA AG) and a reverse primer which included Xho I and Not I restriction sites (CTC GAG CGG CCG CAC GCA GTC AGA TCT GAT ATT C). The product was subcloned into pcDNA3.1(+) (Invitrogen).

Tissue expression by Northern blots and expression assays were performed as described above. These experiments indicated that mRNA for the human hCCh4 channel protein reported herein has a significantly different distribution of expression compared to rabbit and rat channel proteins reported in the art (see, e.g., Hoenderop et al., supra, and Peng et al., supra). Thus, whereas the rabbit EcaC protein of Hoenderop is expressed in high levels in the kidney and the rat CaT1 protein of Peng is expressed in the lining of the gut, and these researchers propose that these proteins mediate Ca²⁺ re-uptake from urine and absorption from the intestinal lumen, respectively, the present experiments indicated that the highest levels of hCCh4 expression occur in the placenta, prostate, salivary gland, and pancreas with lower but significant levels observed in a number of brain regions including cerebral cortex, nucleus accumbens, caudate nucleus, putamen, hippocampus, medulla, spinal cord, pons, corpus callosum, substantia nigra, thalamus, and others. While hCCh4 transcripts are also observed in kidney and various segments of the gut, the levels of expression in the these tissues were much reduced compared to the rabbit and rat proteins disclosed in the art.

In addition, using the Ca²⁺-flux assay described above, it was determined that hCCh4, like hCCh3, does not confer sensitivity to capsaicin or resiniferatoxin and thus the novel human channel encoded by hCCh4 does not appear to mediate its actions via capsaicin or other related vanilloids.

### DEPOSIT OF MICROORGANISMS

The following microorganisms were deposited with the American Type Culture Collection (ATCC), 10801 University Blvd., Manassas, Va. 20110 on Jan. 13, 2000 and assigned the following numbers:

| Microorganism | ATCC Deposit No. |
|---|---|
| hCCh3.1-pcDNA3.1(+) | PTA-1204 |
| hCCh3.2-pcDNA3.1(+) | PTA-1205 |
| hCCh4-pcDNA3.1(+) | PTA-1206 |

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. Various publications are cited herein, the disclosures of which are incorporated by reference in their entireties.

We claim:

1. An isolated nucleic acid comprising:
(a) a nucleic acid sequence that encodes a polypeptide having the amino acid sequence of FIG. 2 or FIG. 4 (SEQ ID NO. ______ );
(b) the complement of the nucleic acid sequences of (a); or
(c) a hCCh3 gene or a complement of a hCCh3 gene as contained in ATCC Deposit Nos. PTA-1204 and PTA-1205.

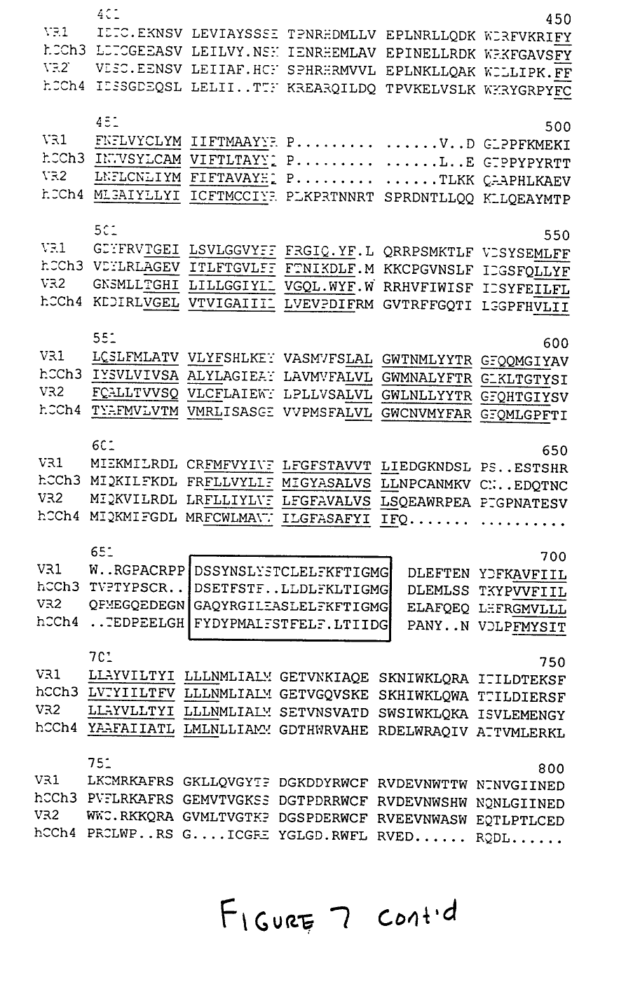

2. An isolated nucleic acid comprising:
(a) a nucleic acid sequence that encodes a polypeptide having the amino acid sequence of FIG. 6 (SEQ ID NO. ______ );
(b) the complement of the nucleic acid of (a); or
(c) a hCCh4 gene or a complement of a hCCh4 gene as contained in ATCC Deposit No. PTA-1206

3. An isolated nucleic acid comprising a nucleic acid capable of hybridizing under stringent conditions to a nucleic acid molecule of claim 1 or 2 and encoding a hCCh polypeptide having an activity of a naturally occurring hCCh polypeptide.

4. An isolated nucleic acid of claim 1 comprising the nucleic acid sequence of FIG. 1.

5. An isolated nucleic acid of claim 1 comprising the nucleic acid sequence of FIG. 3.

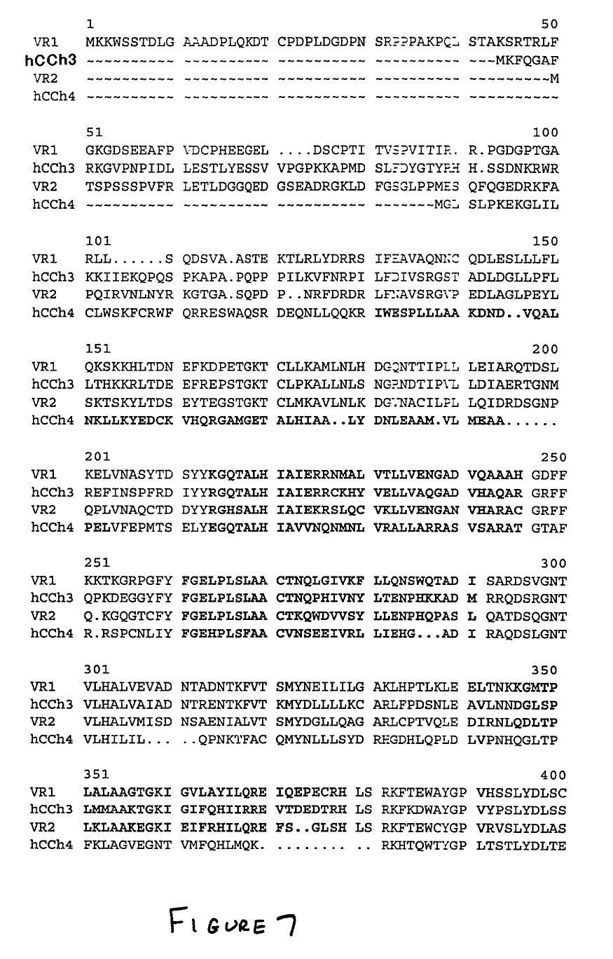

6. An isolated nucleic acid of claim 2 comprising the nucleic acid sequence of FIG. 5.

7. An isolated nucleic acid of claim 1 or 2, which is genomic or cDNA.

8. An isolated nucleic acid of claim 1 or 2, which is RNA.

9. An isolated nucleic acid of claim 1 or 2 further comprising a label.

10. A recombinant vector comprising a nucleic acid of claim 1, 2, 4, 5, or 6.

11. A recombinant vector comprising a nucleic acid of claim 3.

12. An expression vector comprising a nucleic acid of claim 1, 2, 4, 5, or 6 operatively associated with a regulatory nucleotide sequence containing transcriptional and translational regulatory information that controls expression of the nucleic acid in a host cell.

13. An expression vector comprising a nucleic acid of claim 3 operatively associated with a regulatory nucleotide sequence containing transcriptional and translational regulatory information that controls expression of the nucleic acid in a host cell.

14. A genetically engineered host cell containing a nucleic acid of claim 1, 2, 4, 5, or 6.

15. A genetically engineered host cell containing a nucleic acid of claim 3.

16. A genetically engineered host cell containing a nucleic acid of claim 1, 2, 4, 5, or 6 operatively associated with a non-native regulatory nucleotide sequence containing transcriptional and translational regulatory information that controls expression of the nucleic acid sequence in a host cell.

17. A genetically engineered host cell containing a nucleic acid sequence of claim 3 operatively associated with a non-native regulatory nucleotide sequence containing transcriptional and translational regulatory information that controls expression of the nucleic acid sequence in a host cell.

18. A method of making an hCCh polypeptide comprising the steps of:
(a) culturing the host cell of claim 16 in an appropriate culture medium to produce an hCCh polypeptide; and
(b) isolating the hCCh polypeptide.

19. A method of making an hCCh polypeptide comprising the steps of:
(a) culturing the host cell of claim 17 in an appropriate culture medium to produce an hCCh polypeptide; and
(b) isolating the hCCh polypeptide.

20. The method of claim 18, wherein the hCCh polypeptide is hCCh3.1, hCCh3.2 or hCCh4 or a functionally equivalent derivative thereof.

21. The method of claim 19, wherein the hCCh polypeptide is hCCh3.1, hCCh3.2 or hCCh4 or a functionally equivalent derivative thereof.

22. An antibody preparation which is specifically reactive with an epitope of an hCCh polypeptide.

23. A substantially pure polypeptide encoded by a nucleic acid of claim 1, 2, 3, 4, 5 or 6.

24. A substantially pure human polypeptide, as depicted in FIG. 2, 4 or 6.

25. A substantially pure polypeptide which is at least 90% identical to the polypeptide as set forth in FIG. 2, 4 or 6.

26. A pharmaceutical composition comprising a therapeutically effective amount of a polypeptide encoded by the nucleic acid claim 1, 2, 3, 4, 5 or 6 and a pharmaceutically acceptable carrier.

27. A pharmaceutical composition comprising a therapeutically effective amount of a human polypeptide of claim 24 and a pharmaceutically acceptable carrier.

28. A pharmaceutical composition comprising a therapeutically effective amount of a human polypeptide of claim 25 and a pharmaceutically acceptable carrier.

29. A method for identifying compounds that modulate hCCh activity comprising:
(a) contacting a test compound to a cell that expresses a hCCh gene;
(b) measuring the level of hCCh gene expression in the cell; and
(c) comparing the level obtained in (b) with the hCCh gene expression obtained in the absence of the compound; such that if the level obtained in (b) differs from that obtained in the absence of the compound, a compound that modulates hCCh activity is identified.

30. A method for identifying compounds that modulate hCCh activity comprising:
(a) contacting a test compound to a cell that contains a hCCh polypeptide;
(b) measuring the level of hCCh polypeptide or activity in the cell; and
(c) comparing the level obtained in (b) with the level of hCCh polypeptide or activity obtained in the absence of the compound; such that if the level obtained in (b) differs from that obtained in the absence of the compound, a compound that modulates hCCh activity is identified.

31. A method for identifying compounds that regulate ion channel-related disorders, comprising:
(a) contacting a test compound with a cell which expresses a nucleic acid of claim 1, 2 or 3, and
(b) determining whether the test compound modulates hCCh activity.

32. A method for identifying compounds that regulate ion channel-related disorders comprising:
(a) contacting a test compound with a nucleic acid of claim 1, 2 or 3; and
(b) determining whether the test compound interacts with the nucleic acid of claim 1, 2 or 3.

33. A method for identifying compounds that regulate ion channel-related disorders, comprising:
(a) contacting a test compound with a cell or cell lysate containing a reporter gene operatively associated with a hCCh regulatory element; and
(b) detecting expression of the reporter gene product.

34. A method for identifying compounds that regulate ion channel-related disorders comprising:
(a) contacting a test compound with a cell or cell lysate containing hCCh transcripts; and
(b) detecting the translation of the hCCh transcript.

35. A method for the treatment of ion channel-related disorders, comprising modulating the activity of a hCCh polypeptide.

36. The method of claim 35, wherein the hCCh polypeptide is hCCh3.1, hCCh3.2 or hCCh4, or a functionally equivalent derivative thereof.

37. The method of claim 35, wherein the method comprises administering an effective amount of a compound that agonizes or antagonizes the activity of the hCCh polypeptide.

38. A method for the treatment of ion channel-related disorders, comprising administering an effective amount of a compound that decreases expression of a hCCh gene.

39. A method for the treatment of ion channel-related disorders, comprising administering an effective amount of a compound that increases expression of a hCCh gene.

40. A pharmaceutical formulation for the treatment of ion channel-related disorders, comprising a compound that activates or inhibits hCCh activity, mixed with a pharmaceutically acceptable carrier.


SEQUENCE LISTING (10 sequences)

SEQ ID NO: 1 - DNA, 2412 bp, Homo sapiens
atgaagttccagggcgccttccgcaagggggtgcccaaccccatcgatctgctggagtccaccctatatgagtcctcggtggtgcctgggcccaagaaagcacccatggactcactgtttgactacggcacctatcgtcaccactccagtgacaacaagaggtggaggaagaagatcatagagaagcagccgcagagccccaaagcccctgcccctcagccgccccccatcctcaaagtcttcaaccggcctatcctctttgacatcgtgtcccggggctccactgctgacctggacgggctgctcccattcttgctgacccacaagaaacgcctaactgatgaggagtttcgagagccatctacggggaagacctgcctgcccaaggccttgctgaacctgagcaatggccgcaacgacaccatccctgtgctgctggacatcgcggagcgcaccggcaacatgcgggagttcattaactcgcccttccgtgacatctactatcgaggtcagacagccctgcacatcgccattgagcgtcgctgcaaacactacgtggaacttctcgtggcccagggagctgatgtccacgcccaggcccgtgggcgcttcttccagcccaaggatgaggggggctacttctactttggggagctgcccctgtcgctggctgcctgcaccaaccagccccacattgtcaactacctgacggagaacccccacaagaaggcggacatgcggcgccaggactgccgaggcaacacagtgctgcatgcgctggtggccattgctgacaacacccgtgagaacaccaagtttgttaccaagatgtacgacctgctgctgctcaagtgtgcccgcctcttccccgacagcaacctggaggccgtgctcaacaacgacggcctctcgcccctcatgatggctgccaagacgggcaagattgggatctttcagcacatcatccggcgggaggtgacggatgaggacacacggcacctgtcccgcaagttcaaggactgggcctatgggccagtgtatccctcgctttatgacctctcctccctggacacgtgtggggaagaggcctccgtgctggagattctggtgtacaacagcaagattgagaaccgccacgagatgctggctgtggagcccatcaatgaactgctgcgggacaagtggcgcaagttcggggccgtctccttctacatcaacgtggtctcctacctgtgtgccatggtcatcttcactctcaccgcctactaccagccgctggagggcacaccgccgtacccttaccgcaccacggtggactacctgcggctggctggcgaggtcattacgctcttcactggggtcctgttcttcttcaccaacatcaaagacttgttcatgaagaaatgccctggagtgaattctctcttcattgatggctccttccagctgctctacttcatctactctgtcctggtgatcgtctcagcagccctctacctggcagggatcgaggcctacctggccgtgatggtctttgccctggtcctgggctggatgaatgccctttacttcacccgtgggctgaagctgacggggacctatagcatcatgatccagaagattctcttcaaggaccttttccgattcctgctcgtctacttgctcttcatgatcggctacgcttcagccctggtctggctcctgaacccgtgtgccaacatgaaggtgtgcaatgaggaccagaccaactgcacagtgcccacttacccctcgtgccgtgacagcgagaccttcagcaccttcctcctggacctgtttaagctgaccatcggcatgggcgacctggagatgctgagcagcaccaagtaccccgtggtcttcatcatcctgctggtgacctacatcatcctcacctttgtgctgctcctcaacatgctcattgccctcatgggcgagacagtgggccaggtctccaaggagagcaagcacatctggaagctgcagtgggccaccaccatcctggacattgagcgctccttccccgtattcctgaggaaggccttccgctctggggagatggtcaccgtgggcaagagctcggacggcactcctgaccgcaggtggtgcttcagggtggatgaggtgaactggtctcactggaaccagaacttgggcatcatcaacgaggacccgggcaagaatgagacctaccagtattatggcttctcgcataccgtgggccgcctccgcagggatcgctggtcctcggtggtaccccgcgtggtggaactgaagaagaactcgaacccggacgaggtggtggtgcctctggacagcatggggaacccccgctgcgatggccaccagcagggttacccccgcaagtggaggactgatgacgccccgctctag

SEQ ID NO: 2 - protein, 792 aa, Homo sapiens
MKFQGAFRKGVPNPIDLLESTLYESSVVPGPKKAPMDSLFDYGTYRHHSSDNKRWRKKIIEKQPQSPKAPAPQPPPILKVFNRPILFDIVSRGSTADLDGLLPFLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKDEGGYFYFGELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGIFQHIIRREVTDEDTRHLSRKFKDWAYGPVYPSLYDLSSLDTCGEEASVLEILVYNSKIENRHEMLAVEPINELLRDKWRKFGAVSFYINVVSYLCAMVIFTLTAYYQPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLLFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFFVDEVNWSHWNQNLGIINEDPGKNETYQYYGFSHTVGRLRRDVVELNKNSNPDEVVVPLDSMGNPRCDGHQQGYPRKWRTDD

SEQ ID NO: 3 - DNA, 2232 bp, Homo sapiens
atgaagttccagggcgccttccgcaagggggtgcccaaccccatcgatctgctggagtccaccctatatgagtcctcggtggtgcctgggcccaagaaagcacccatggactcactgtttgactacggcacctatcgtcaccactccagtgacaacaagaggtggaggaagaagatcatagagaagcagccgcagagccccaaagcccctgcccctcagccgccccccatcctcaaagtcttcaaccggcctatcctctttgacatcgtgtcccggggctccactgctgacctggacgggctgctcccattcttgctgacccacaagaaacgcctaactgatgaggagtttcgagagccatctacggggaagacctgcctgcccaaggccttgctgaacctgagcaatggccgcaacgacaccatccctgtgctgctggacatcgcggagcgcaccggcaacatgcgggagttcattaactcgcccttccgtgacatctactatcgaggtcagacagccctgcacatcgccattgagcgtcgctgcaaacactacgtggaacttctcgtggcccagggagctgatgtccacgcccaggcccgtgggcgcttcttccagcccaaggatgaggggggctacttctactttggggagctgcccctgtcgctggctgcctgcaccaaccagccccacattgtcaactacctgacggagaacccccacaagaaggcggacatgcggcgccaggactcgcgaggcaacacagtgctgcatgcgctggtggccattgctgacaacacccgtgagaacaccaagtttgttaccaagatgtacgacctgctgctgctcaagtgtgcccgcctcttccccgacagcaacctggaggccgtgctcaacaacgacggcctctcgcccctcatgatggctgccaagacgggcaagattgggaaccgccacgagatgctggctgtggagcccatcaatgaactgctgcgggacaagtggcgcaagttcggggccgtctccttctacatcaacgtggtctcctacctgtgtgccatggtcatvttcactctcaccgcctactaccagccgctggagggcacaccgccgtacccttaccgcaccacggtggactacctgcggctggctggcgaggtcattacgctcttcactggggtcctgttcttcttcaccaacatcaaagacttgttcatgaagaaatgccctggagtgaattctctcttcattgatggctccttccagctgctctacttcatctactctgtcctggtgatcgtctcagcagccctctacctggcagggatcgaggcctacctggccgtgatggtctttgccctggtcctgggctggatgaatgccctttacttcacccgtgggctgaagctgacggggacctatagcatcatgatccagaagattctcttcaaggaccttttccgattcctgctcgtctacttgctcttcatgatcggctacgcttcagccctggtctccctcctgaacccgtgtgccaacatgaaggtgtgcaatgaggaccagaccaactgcacagtgcccacttacccctcgtgccgtgacagcgagaccttcagcaccttcctcctggacctgtttaagctgaccatcggcatgggcgacctggagatgctgagcagcaccaagtaccccgtggtcttcatcatcctgctggtgacctacatcatcctcacctttgtgctgctcctcaacatgctcattgccctcatgggcgagacagtgggccaggtctccaaggagagcaagcacatctggaagctgcagtgggccaccaccatcctggacattgagcgctccttccccgtattcctgaggaaggccttccgctctggggagatggtcaccgtgggcaagagctcggacggcactcctgaccgcaggtggtgcttcagggtggatgaggtgaactggtctcactggaaccagaacttgggcatcatcaacgaggacccgggcaagaatgagacctaccagtattatggcttctcgcataccgtgggccgcctccgcagggatcgctggtcctcggtggtaccccgcgtggtggaactgaagaagaactcgaacccggacgaggtggtggtgcctctggacagcatggggaacccccgctgcgatggccaccagcagggttacccccgcaagtggaggactgatgacgccccgctctag

SEQ ID NO: 4 - protein, 734 aa, Homo sapiens
MKFQGAFRKGVPNPIDLLESTLYESSVVPGPKKAPMDSLFDYGTYRHHSSDNKRWRKKIIEDQPQSPKAPAPQPPPILKVFNRPILFDIVSRGSTADLDGLLPFLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGNRHEMLAVEPINELLRDKWRKFGAVSFYINVVSYLCAMVIFTLTAYYQPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLLFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKNETYQYYGFSHTVGRLRRFRWSSVVPRVVELNKNSNPDEVVVPLDSMGNPRCDGHQQGYPRKWRTDDAPL

SEQ ID NO: 5 - DNA, 2178 bp, Homo sapiens
atgggtttgtcactgcccaaggagaaagggctaattctctgcctatggagcaagttctgcagatggttccagagacgggagtcctgggcccagagccgagatgagcagaacctgctgcagcagaagaggatctgggagtctcctctccttctagctgccaaagataatgatgtccaggccctgaacaagttgctcaagtatgaggattgcaaggtgcaccagagaggagccatgggggaaacagcgctacacatagcagccctctatgacaacctggaggccgccatggtgctgatggaggctgccccggagctggtctttgagcccatgacatctgagctctatgagggtcagactgcactgcacatcgctgttgtgaaccagaacatgaacctggtgcgagccctgcttgcccgcagggccagtgtctctgccagagccacaggcactgccttccgccgtagtccctgcaacctcatctactttggggagcaccctttgtcctttgctgcctgtgtgaacagtgaggagatcgtgcggctgctcattgagcatggagctgacatccgggcccaggactccctgggaaacacagtgttacacatcctcatcctccagcccaacaaaacctttgcctgccagatgtacaacctgttgctgtcctacgacagacatggggaccacctgcagcccctggacctcgtgcccaatcaccagggtctcacccctttcaagctggctggagtggagggtaacactgtgatgtttcagcacctgatgcagaagcggaagcacacccagtggacgtatggaccactgacctcgactctctatgacctcacagagatcgactcctcaggggatgagcagtccctgctggaacttatcatcaccaccaagaagcgggaggctcgccagatcctggaccagacgccggtgaaggagctggtgagcctcaagtggaagcggtacgggcggccgtacttctgcatgctgggtgccatatatctgctgtacatcatctgcttcaccatgtgctgcatctaccgccccctcaagcccaggaccaataaccgcacgagcccccgggacaacaccctcttacagcagaagctacttcaggaagcctacatgacccctaaggacgatatccggctggtcggggagctggtgactgtcattggggctatcatcatcctgctggtagaggttccagacatcttcagaatgggggtcactcgcttctttggacagaccatccttgggggcccattccatgtcctcatcatcacctatgccttcatggtgctggtgaccatggtgatgcggctcatcagtgccagcggggaggtggtacccatgtcctttgcactcgtgctgggctggtgcaacgtcatgtacttcgcccgaggattccagatgctaggccccttcaccatcatgattcagaagatgatttttggcgacctgatgcgattctgctggctgatggctgtggtcatcctgggctttgcttcagccttctatatcatcttccagacagaggaccccgaggagctaggccacttctacgactaccccatggccctgttcagcaccttcgagctgttccttaccatcatcgatggcccagccaactacaacgtggacctgcccttcatgtacagcatcacctatgctgcctttgccatcatcgccacactgctcatgctcaacctcctcattgccatgatgggcgacactcactggcgagtggcccatgagcgggatgagctgtggagggcccagattgtggccaccacggtgatgctggagcggaagctgcctcgctgcctgtggcctcgctccgggatctgcggacgggagtatggcctgggagaccgctggttcctgcgggtggaagacaggcaagatctcaaccggcagcggatccaacgctacgcacaggccttccacacccggggctctgaggatttggacaaagactcagtggaaaaactagagctgggctgtcccttcagcccccacctgtcccttcctatgccctcagtgtctcgaagtacctcccgcagcagtgccaattgggaaaggcttcggcaagggaccctgaggagagacctgcgtgggataatcaacaggggtctggaggacggggagagctgggaatatcagatctga

SEQ ID NO: 6 - protein, 725 aa, Homo sapiens
MGLSLPKEKGLILCLWSKFCRWFQRRESWAQSRDEQNLLQQKRIWESPILLAAKDNDVQALNKLLKYEDCKVHQRGAMGETALHIAALYDNLEAAMVLMEAAPELVFEPMTSELYEGQTALHIAVVNQNMNLVRALLARRASVSARATGTAFRRSPCNLIYFGEHPLSFAACVNSEEIVRLLIEHGADIRAQDSLGNTVLHILILQPNKTFACQMYNLLLSYDRHGDHLQPLDLVPNHQGLTPFKLAGVEGNTVMFQHLMQKRKHTQWTYGPLTSTLYDLTEIDSSGDEQSLLESIITTKKREARQILDQTPVKELVSLKWKRYGRPYFCMLGAIYLLYIICFTMCCIYRPLKPRTNNRTSPRDNTLLQQKLLQEAYMTPKDDIRLVGELVTVIGAIIILLVEVPDIFRMGVTRFFGQTILGGPFHVLIITYAFMVLVTMVMRLISASGEVVPMSFALVLGWCNVMYFARGFQMLGPFTIMIQKMIFGDLMRFCWLMAVVILGFASAFYIIFQTEDPEELGHFYDYPMALFSTFELFLTIIDGPANYNVDLPFMYSITYAAFAIIATLLMLNLLIAMMGDTHWRVAHERDELWRAQIVATTVMLERKLPRCLWPRSGICGREYGLGDRWFLRVEDRQDLNRQRIQRYAQAFHTRGSEDLDKDSVEKLELGCPFSPHLSLPMPSVSRSTSRSSANWERLRQGTLRRDLRGIINRGLEDGESWEYQI

SEQ ID NO: 7 - protein, 829 aa, Homo sapiens
MKKWSSTDLGAAADPLQKDTCPDPLDGDPNSRPPPAKPQLSTAKSRTRLFGKGDSEEAFPVDCPHEEGELDSCPTITVSPVITIRRPGDGPTGARLLSQDSVAASTEKTLRLYDRRSIFEAVAQNNCDLESLLLFLQKSKKHLTDNEFKDPETGKTCLLKAMLNLHDGQNTTIPLLLEIARQTDSLKELVNASYTDSYYKGQTALHIAIERRNMALVTLLVENGADVQAAAHGDFFKKTKGRPGFYFGELPLSLAACTNQLGIVKFLLQNSWQTADISARDSVGNTVLHALVEVADNTADNTKFVTSMYNEILILGAKLHPTLKLEELTNKKGMTPLALAAGTGKIGVLAYILQREIQEPECRHLSRKFTEWAYGPVHSSLYDLSCIDTCEKNSVLEVIAYSSSETPNRHDMLLVEPLNRLLQDKWDRFVKRIFYFNELVYCLYMIIFTMAAYYRPVDGLPPFKMEKIGDYFRVTGEILSVLGGVYFFFRGIQYFLQRRPSMKTLFFDSYSEMLFFLQSLFMLATVVLASMVFSLALGWTNMLYYTRGFQQMGIYAVMIEKMILRDLCRFMFVYIVFLFGFSTAVVTLIEDGKNDSLPSESTSHRWRGPACRPPDSSYNSLYSTCLELFKFTIGMGDLEFTENYDFKAVFIILLLAYVILTYILLLNMLIALMGETVNKIAQESKNIWKLQRAIFILDTEKSFLKCMRKAFRSGKLLQVGYTPDGKDDYRWCFRVDEVNWTTWNTNVGIINEDPGNCEGVKRTLSFSLRSSRVSGRHWKNFALVPLLREASARDRQSAQPEEVYLRQFSGSLKPEDAEVFKSPAASGEK

SEQ ID NO: 8 - protein, 803 aa, Homo sapiens
MKFQGAFRKGVPNPIDLLESTLYESSVVPGPKKAPMDSLFDYGTYRHHSSDNKRWRKKIIEKQPQSPKAPAPQPPPILKVFNRPILFDIVSRGSTADLDGLLPFLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKDEGGYFYFGELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGIFQHIIRREVTDEDTRHLSRKFKDWAYGPVYPSLYDLSSLDTCGEEASVLEILVYNSKIENRHEMLAVEPINELLRDKWRKFGAVSFYINVVSYLCAMVIFTLTAYYIPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLIFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKNETYQYYGFSHTVGRLRRDRWSSVVPRVVELNKNSNPDEVVVPLDSMGNPRCDGHQQGYPRKWRTDDAPL

SEQ ID NO: 9 - protein, 760 aa, Homo sapiens
MTSPSSSPVFRLETLDGGQEDGSEADRGKLDFGSGLPPMESQFQGEDRKFAPQIRVNLNYRKGTGASQPDPNRFDRDRLFNAVSRGVPEDLAGLPEYLSKTSKYLTDSEYTEGSTGKTCLMKAVLNLKDGVNACILPLPQIDRDSGNPQPLVNAQCTDDYYRGHSALHIAIEKRSLQCVKLLVENGANVHARACGRFFQKGQGTCFYFGELPLSLAACTKQWDVVSYLLENPHQPASLQATDSQGNTVLHALVMISDNSAENIALVTSMYDGLLQAGARLCPTVQLEDIRNLQDLTPLKLAAKEGKIEIFRHILQREFSGLSHLSRKFTEWCYGPVRVSLYDLASVESCEENSVLEIIAFHCKSPHRHRMVVLEPLNKLLQAKWILLIPKFFLNFLCNLIYMFIFTAVAYHIPTLKKQAAPHLKAEVGNSMLLTGHILILLGGIYLLVGQLWRHVFIWISFIISYFEILFLFQALLTVVSQVLCFLAIEWYLPLLVSALVLGWLNLLYYTRGFQHTGIYSVMIQKVILRDLLRFLLIYLVFLFGFAVALVSLSQEAWRPEAPTGPNATESVQPMEGQEDEGNGAQYRGILEASLELFKFTIGMGELAFQEQLHFRGMVLLLLLAYVLLTYILLLNMLIALMSETVNSVATDSWSIWKLQKAISVLEMENGYWWCRKKQRAGVMLTVGTKPDGSPDERWCFRVEEVNWASWEQTLPTLCEDPSGAGVPRTLENPVLASPPKEDEDGASEENYVPVQLLQSN

SEQ ID NO: 10 - protein, 425 aa, Homo sapiens
MGLSLPKEKGLILCLWSKFCRWFQRRESWAQSRDEQNLLQQKRIWESPLLLAAKDNDVQALNKLLKYEDCKVHQRGAMGETALHIAALYDNLEAAMVLMEAAPELVFEPMTSELYEGQTALHIAVVNQNMNLVRALLARRASVSARATGTAFRRSPCNLIYFGEHPLSFAACVNSEEIVRLLIEHGADIRAQDSLGNTVLHILILQPNKTFACQMYNLLLSYDRHGDHLQPLDLVPNHQGLTPFKKLAGVEGNTVMFQHLMQKRKHTQWTYGPLTSTLYDLTEIDSSGDEQSLLELIITTKKREARQILDQTPVKELVSLKWKRYGRPYFCMLGAIYLLYIICFTMCCIYRPLKPRTNNRTSPRDNTLLQQKLLQEAYMTPKDDIRLVGELVTVIGAIIIDIFRMGVTRFFGQTILGGPFHVLII